(19) United States
(12) Patent Application Publication
(10) Pub. No.: US 2013/0109055 A1
(43) Pub. Date: May 2, 2013

(54) USE OF MAMMALIAN PROMOTERS IN FILAMENTOUS FUNGI

(71) Applicant: BP Corporation North America Inc., Houston, TX (US)

(72) Inventors: Nicholas J. Ryding, San Diego, CA (US); Wenqi Hu, San Diego, CA (US); Biyu Li, San Diego, CA (US)

(73) Assignee: BP Corporation North America Inc., Houston, TX (US)

(21) Appl. No.: 13/665,609

(22) Filed: Oct. 31, 2012

Related U.S. Application Data
(60) Provisional application No. 61/553,901, filed on Oct. 31, 2011.

(51) Int. Cl. C12N 15/80

(52) U.S. Cl. CPC C12N 15/80
USPC 435/69.1; 435/320.1; 435/254.11; 435/254.3; 435/254.7; 435/254.8; 435/254.4; 435/254.5; 435/254.6; 435/209; 435/99; 435/162

(57) ABSTRACT

The present disclosure is directed to the use of mammalian promoters to drive recombinant expression in filamentous fungal cells. In certain aspects, the present disclosure provides an expression cassette useful for the expression of polypeptide in filamentous fungal cells. Also provided herein, are vectors and recombinant filamentous fungal cells comprising the expression cassettes of the present disclosure, and methods of making and using the same for recombinant polypeptide expression.

### 1. BACKGROUND

The use of recombinant expression has greatly simplified the production of large quantities of commercially valuable proteins. Currently, there is a varied selection of expression systems from which to choose for the production of any given protein, including prokaryotic and eukaryotic hosts. A variety of gene expression systems have been developed for use with filamentous fungal cells. Many systems entail the use of inducible promoters, the majority of which require the addition of an exogenous inducer molecule to the culture which is cost prohibitive in large scale commercial fermentations, or endogenous promoters that are susceptible to regulation by endogenous filamentous fungal proteins. Thus, there is a need for expression systems that are economically viable and provide robust expression in large scale commercial fermentations.

### 2. SUMMARY

The present disclosure relates to the use of heterologous promoters to drive recombinant polypeptide expression in filamentous fungi. More particularly, the present disclosure relates to the use of promoters that are operable in mammalian cells to drive recombinant polypeptide expression in filamentous fungi. The present disclosure is based, in part, on applicants' discovery that promoters that are constitutively active in mammalian cells are capable of eliciting high expression levels in filamentous fungi such as Trichoderma reesei, particularly when the 5′ UTR sequence normally associated with the promoter is replaced by a filamentous fungal 5′ UTR sequence. Thus, the present disclosure relates to recombinant filamentous fungal expression systems utilizing promoters operable in mammalian cells, which are preferably constitutive promoters. Such promoters can be derived from a mammalian genome or the genome of a mammalian virus, and are collectively referred to herein as “mammalian promoters.”

Thus, the present disclosure provides expression cassettes comprising a mammalian promoter operably linked to a coding sequence for a polypeptide of interest (a “POI”). Mammalian promoters that are suitable for recombinant expression in filamentous fungi include, but are not limited to, the cytomegalovirus (CMV) promoter. Additional promoters suitable for practicing the present invention are described in Section 4.1.1.

The sequence encoding the POI can be from a prokaryotic (e.g., bacterial), eukaryotic (e.g., plant, filamentous fungal, yeast or mammalian) or viral source. It can optionally include introns. In some embodiments, the polypeptide coding sequence comprises a signal sequence, which directs the POI to be secreted by the filamentous fungal cell. In a specific exemplary embodiment, the polypeptide coding sequence is a polypeptide coding sequence of a Cochliobolus heterostrophus β-glucosidase gene. Further POIs are described in Section 4.1.3.

In order to achieve robust expression of the POI from the mRNA transcript, the expression cassette preferably includes a sequence that corresponds to a 5′ untranslated region (5′ UTR) in the mRNA resulting from transcription of the expression cassette (for convenience referred to as a “5′ UTR” in the expression cassette). A 5′ UTR can contain elements for controlling gene expression by way of regulatory elements. It begins at the transcription start site and ends one nucleotide (nt) before the start codon of the coding region. A 5′ UTR that is operable in a filamentous fungal cell can be included in the expression cassettes of the disclosure. The source of the 5′ UTR can vary provided it is operable in the filamentous fungal cell. In various embodiments, the 5′ UTR can be derived from a yeast gene or a filamentous fungal gene. The 5′ UTR can be from the same species one other component in the expression cassette (e.g., the promoter or the polypeptide coding sequence), or from a different species than the other component. The 5′ UTR can be from the same species as the filamentous fungal cell that the expression construct is intended to operate in. By of example and not limitation, the 5′ UTR can from a Trichoderma species, such as Trichoderma reesei. In an exemplary embodiment, the 5′ UTR comprises a sequence corresponding to a fragment of a 5′ UTR from a T reesei glyceraldehyde-3-phosphate dehydrogenase (gpd). In a specific embodiment, the 5′ UTR is not naturally associated with the CMV promoter. Additional 5′ UTRs are described in Section 4.1.2.

For effective processing of the transcript encoding the POI, the expression cassette further includes a sequence that corresponds to a 3′ untranslated region (3′ UTR) in the mRNA resulting from transcription of the expression cassette (for convenience referred to as a “3′ UTR” in the expression cassette). A 3′ UTR minimally includes a polyadenylation signal, which directs cleavage of the transcript followed by the addition of a poly(A) tail that is important for the nuclear export, translation, and stability of mRNA. As with the 5′ UTR, the 3′ UTR can be derived from a yeast gene or a filamentous fungal gene. Additional 3′ UTR are described in Section 4.1.4.

Accordingly, in certain aspects, as illustrated in FIG. 1, the present disclosure provides expression cassettes comprising, operably linked to 5′ and to 3′ direction: (1) a mammalian promoter, (2) a 5′ UTR (i.e., a sequence coding for a 5′ UTR), (3) a coding sequence for a POI, and (4) a 3′ UTR (i.e., a coding sequence for a 3′ UTR). Each of these components is described below and in the corresponding sub-section of Section 4.1.

The expression cassettes of the disclosure can encode more than one POI (e.g., a first POI, a second POI, and optionally a third or more POIs). In embodiments where the expression cassette comprises more than one polypeptide coding sequence, the expression cassette can include an internal ribosome binding entry site (“IRES”) sequence between the POI coding sequences.

The present disclosure further provides filamentous fungal cells engineered to contain an expression cassette. Recombinant filamentous fungal cells may be from any species of filamentous fungus. In some embodiments, the filamentous fungal cell is a Trichoderma sp., e.g. Trichoderma reesei. The expression cassette can be extra-genomic or part of the filamentous fungal cell genome. One, several, or all components in an expression cassette can be introduced into a filamentous fungal cell by one or more vectors. Accordingly, the present disclosure also provides vectors comprising expression cassettes or components thereof (e.g., a promoter). The vectors can also include targeting sequences that are capable of directing integration of the expression cassette or expression cassette component into a filamentous cell by homologous recombination. For example, the vector can include a mammalian promoter flanked by sequences corresponding to a filamentous fungal gene encoding a POI such that upon transformation of the vector into a filamentous fungal cell the flanking sequences will direct integration of the promoter sequence into a location of the filamentous fungal genome where it is operably linked to the POI coding sequence and directs recombinant expression of the POI.

The present disclosure further provides vectors comprising, operably linked in a 5′ to 3′ direction, a mammalian promoter, a 5′ UTR sequence, one or more unique restriction sites, and a 3′ UTR. The unique restriction sites facilitate cloning of any POI coding sequence into the vector to generate an expression cassette of the disclosure.

The vectors are typically capable of autonomous replication in a prokaryotic (e.g., E. coli) and/or eukaryotic (e.g., filamentous fungal) cells and thus contain an origin of replication that is operable in such cells. The vectors preferably include a selectable marker, such as an antibiotic resistance marker or an auxotrophy marker, suitable for selection in prokaryotic or eukaryotic cells.

Methods of making the recombinant filamentous fungal cells described herein include methods of introducing vectors comprising expression cassettes or components thereof into filamentous fungal cells and, optionally, selecting for filamentous fungal cells whose genomes contain an expression cassette of the disclosure (for example by integration of a entire expression cassette or a portion thereof). Such methods are described in more detail in Section 4.4 below and in the Examples.

Also provided herein are methods of using the recombinant filamentous fungal cells described herein to produce a POI. Generally, the methods comprise culturing a recombinant filamentous fungal cell comprising an expression cassette of the disclosure under conditions that result in expression of the POI. Optionally, the methods can further include a step of recovering the POI from cell lysates or, where a secreted POI is produced, from the culture medium. The method can further comprise additional protein purification or isolation steps, as described below in Section 4.6.

The recombinant filamentous fungal cells of the disclosure can be used to produce cellulase compositions. Where the production of cellulase compositions (including whole cellulase compositions and fermentation broths) is desired, the recombinant filamentous fungal cells can be engineered to express as POIs one or more cellulases, hemicellulases and/or accessory proteins. Exemplary cellulases, hemicellulases and/or accessory proteins are described in Section 4.1.3. The cellulase compositions can be used, inter alia, in processes for saccharifying biomass. Additional details of saccharification reactions, and additional applications of the variant β-glucosidase polypeptides, are provided in Section 4.6.

All publications, patents, patent applications, GenBank sequences, Accession numbers, and ATCC deposits, cited herein are hereby expressly incorporated by reference for all purposes.

### 3. BRIEF DESCRIPTION OF THE FIGURES

FIG. 1 provides a schematic drawing of an expression cassette comprising (1) a promoter, (2) a 5′ untranslated region (5′ UTR), (3) a coding sequence, with or without introns, and (4) a 3′ untranslated region (3′ UTR).

FIGS. 2A-2C provide schematic drawings of an extra-genomic expression cassette (FIG. 2A), a genomic expression cassette (FIG. 2B), and integration of expression cassette components into the genome of a filamentous fungal cell to generate a genomic expression cassette (FIG. 2C).

FIG. 3 illustrates a vector, referred to as pC, comprising a mammalian viral promoter from cytomegalovirus (CMV) and the terminator of Trichoderma reesei CBHI gene, which includes a 3′ UTR. pC includes unique restriction sites between the 5′ and 3′ UTR sequences (SpeI, FseI, BamHI, SbfI), into which the POI coding sequence(s) can be cloned, and a selectable marker gene, pyr4.

FIG. 4 provides a micrograph mapping the promoter and coding regions for Trichoderma reesei glyceraldehyde-3-phosphate dehydrogenase (gpd), showing DNA fragments corresponding to nucleotide sequences in Trichoderma reesei glyceraldehyde-3-phosphate dehydrogenase (gpd) cDNA or genomic DNA produced by PCR using nested primers specific to sequences from 34 to 443 bp upstream of the gpd translation start site.

FIGS. 5A-5D provide schematic maps of expression vectors comprising a mammalian viral promoter, a 5′ UTR, a polypeptide of interest, and a terminator sequence that includes a 3′ UTR. FIG. 5A illustrates a vector, referred to as pC-UTR, comprising a CMV promoter, a 5′UTR sequence corresponding to the native CMV 5′UTR (CMV native UTR), and a polypeptide coding sequence of a Cochliobolus heterostrophus β-glucosidase gene, a terminator sequence from the Trichoderma reesei CBHI gene, which includes a 3′ UTR, and a selectable marker (pyr). FIG. 5B illustrates a vector, referred to as pC-100, comprising a CMV promoter, a 5′UTR sequence corresponding to 100 base pairs (bp) sequence from the 5′UTR of the Trichoderma reesei glyceraldehyde-3-phosphate dehydrogenase (gpd) gene (100 bp 5′ UTR from gpd), a polypeptide coding sequence of a Cochliobolus heterostrophus β-glucosidase gene, a terminator sequence from the Trichoderma reesei CBHI gene, which includes a 3′ UTR, and a selectable marker (pyr). FIG. 5C illustrates a vector, referred to as pC-150, comprising a CMV promoter, a 5′ UTR sequence corresponding to 150 base pairs (bp) sequence from the 5′ UTR of the Trichoderma reesei glyceraldehyde-3-phosphate dehydrogenase (gpd) gene (150 bp 5′ UTR from gpd), a polypeptide coding sequence of a Cochliobolus heterostrophus β-glucosidase gene, a terminator sequence from the Trichoderma reesei CBHI gene, which includes a 3′ UTR, and a selectable marker (pyr). FIG. 5D illustrates a vector, referred to as pC-200, comprising a CMV promoter, a 5′ UTR sequence corresponding to 200 base pairs (bp) sequence from the 5′ UTR of the Trichoderma reesei glyceraldehyde-3-phosphate dehydrogenase (gpd) gene (200 bp 5′ UTR from gpd), a polypeptide coding sequence of a Cochliobolus heterostrophus β-glucosidase gene, a terminator sequence from the Trichoderma reesei CBHI gene, which includes a 3′ UTR, and a selectable marker (pyr).

FIG. 6A-B provides a graph of β-glucosidase activity (in relative units) in 7 separate isolates of a Trichoderma reesei strain MCG80 transformed with one of pC-UTR, pC-100, pC-150, or pC-200, compared to isolates of the parent Trichoderma reesei strain transformed with a vector carrying a selectable marker but without an expression cassette (MCG80pyr4+). FIG. 6A provides results for strains tested in Aspergillus Complete Medium. FIG. 6B provides results for strains tested in Complete Medium.

FIG. 7 shows the increase in β-glucosidase activity following fermentation of a Trichooderma reesei strain containing a single chromosomally integrated copy of the pC-200 plasmid, which comprises a CMV promoter, a 5′ UTR sequence corresponding to 200 base pairs (bp) sequence from the 5′ UTR of the Trichoderma reesei glyceraldehyde-3-phosphate dehydrogenase (gpd) gene (200 bp 5′ UTR from gpd), a polypeptide coding sequence of a Cochliobolus heterostrophus β-glucosidase gene, a terminator sequence from the Trichoderma reesei CBHI gene, which includes a 3′ UTR, and a selectable marker (pyr).

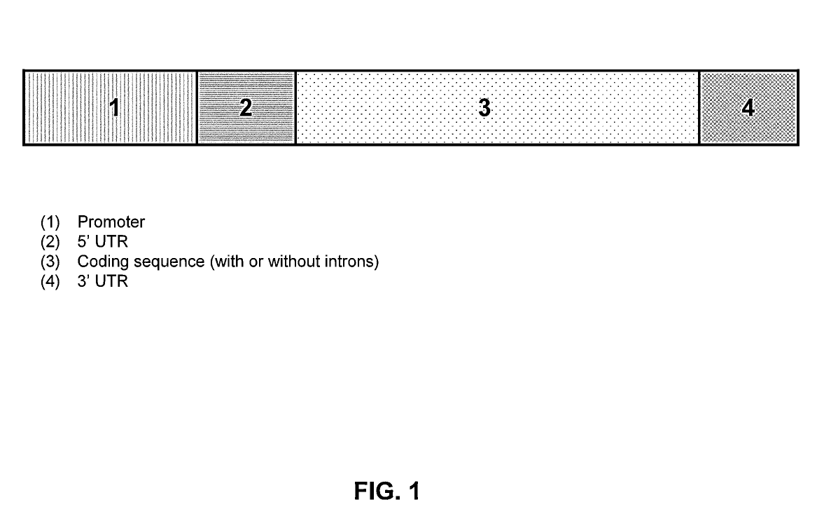

### 4. DETAILED DESCRIPTION

Applicants have discovered that promoters that are active in mammals are useful for expressing genes of interest in filamentous fungi and that, when combined with 5′ untranslated regions (5′ UTR), can significantly increase the yield of active polypeptide expressed in a filamentous fungal cell. Consequently, provided herein, are expression cassettes comprising four components, operably linked in a 5′ to 3′ direction: a promoter that is active in a mammal, a 5′ UTR, a polypeptide coding sequence, and a 3′ UTR. These expression cassettes, described in more detail below, can be transformed into filamentous fungal cells and permit the production and recovery of polypeptides of interest. Accordingly, the present disclosure provides expression cassettes, vectors comprising expression cassettes or components thereof, filamentous fungal cells bearing expression cassettes, and methods of producing, recovering and purifying polypeptides of interest from the filamentous fungal cells described herein.

4.1. Expression Cassette

The expression cassette of the present disclosure typically comprises, operably linked in a 5′ to 3′ direction: (a) a promoter active in a plant, (b) a 5′ untranslated region, (c) a coding sequence, and (4) a 3′ untranslated region, features and examples of which are described further herein below.

4.1.1. Promoter Sequences

The promoters useful in the expression cassettes described herein are promoters that are active in mammalian cells. The promoter can be a mammalian promoter, i.e., a promoter that is native to a mammalian genome, or a promoter from a mammalian virus. Collectively they are referred to herein as “mammalian promoters.”

The mammalian promoters are preferably strong constitutive promoters, e.g., promoters that have at least 20% of the activity of the T reesei CBHI promoter in a filamentous fungus such as T reesei. Promoter activity can be assayed by comparing reporter protein (e.g., green fluorescent protein (“GFP”)) production by filamentous fungal cells (e.g., T. reesei cells) transformed with a vector (e.g., pW as described in the Examples below) containing the test promoter operably linked to the reporter protein coding sequence (the “test vector”) relative to filamentous fungal cells transformed with vector in which the test promoter is substituted with the CBHI promoter (the “control” vector). Reporter protein expression is measured or compared in filamentous fungal cells transformed with the test vector and in filamentous fungal cells transformed with the control vector grown under suitable growth conditions, e.g., in minimal medium containing 2% lactose as described in Murray et al., 2004, Protein Expression and Purification 38:248-257 and Ilmen et al., 1997, Appl. Environmental Microbiol. 63(4):1298-1306. The promoter of interest is considered to be a strong promoter if reporter protein expression in filamentous fungal cells transformed with the test vector is at least about 20% the level of reporter expression observed in the filamentous fungal cells transformed with the control vector. A promoter that can be used in accordance with the present disclosure can, in specific embodiments, have at least 20%, at least 30%, at least 40%, at least 50%, at least 60%, or at least 75% the activity of the CBHI promoter in the assay described above.

Mammalian viral genes are often highly expressed and have a broad host range; therefore sequences encoding mammalian viral genes provide particularly useful promoter sequences. Promoters useful in the expression cassettes provided herein include mammalian viral promoters. Such promoters can be from any family of mammalian virus, including but not limited to viruses belong to one of the Retroviridae, Picornaviridae, Calciviridae, Togaviridae, Flaviridae, Coronaviridae, Rhabdoviridae, Filoviridae, Paramyxoviridae, Orthomyxoviridae, Bungaviridae, Arenaviridae, Reoviridae, Birnaviridae, Hepadnaviridae, Parvoviridae, Papovaviridae, Adenoviridae, Herpesviridae, Polyomaviridae, Poxyiridae and Iridoviridae families. In some embodiments, however, the mammalian virus is not a member of the Polyomaviridae family.

Specific examples of mammalian viral promoters include those derived from the Rous sarcoma virus (RSV) long terminal repeat (LTR) (see, e.g., Yamamoto et al., 1980, Cell 22:787-797), the cytomegalovirus immediate early gene (CMV), the SV40 early promoter (Benoist and Chambon, 1981, Nature 290:304-310), the adenovirus major late promoter, the mouse mammary tumor virus LTR, and the herpes thymidine kinase gene (see, e.g., Wagner et al., 1981, Proc. Natl. Acad. Sci. U.S.A. 78:1441-1445).

In addition, sequences derived from non-viral genes, such as the human β-actin promoter (ACTB) gene, the elongation factor-1α (EF1α) gene, the phosphoglycerate kinase (PGK) gene, the ubiquitinC (UbC) gene, and the murine metallotheionin gene, also provide useful promoter sequences.

The presence of an enhancer element (enhancer) will usually increase expression levels. An enhancer is a regulatory DNA sequence that can stimulate transcription up to 1000-fold when linked to homologous or heterologous promoters, with synthesis beginning at the normal RNA start site. Enhancer elements derived from viruses may be particularly useful, because they usually have a broader host range. Examples include the SV40 early gene enhancer (Dijkema et al., 1985, EMBO J. 4:761) and the enhancer/promoters derived from the long terminal repeat (LTR) of the Rous Sarcoma Virus (Gorman et al., 1982, Proc. Natl. Acad. Sci. 79:6777) and from human cytomegalovirus (Boshart et al., 1985, Cell 41:521). Additionally, some enhancers are regulatable and become active only in the presence of an inducer, such as a hormone or metal ion (Sassone-Corsi and Borelli, 1986, Trends Genet. 2:215; Maniatis et al., 1987, Science 236:1237).

In certain aspects, the promoter is a CMV promoter comprising a nucleotide sequence corresponding to SEQ ID NO:1, or a promoter comprising a nucleotide sequence having at least 70%, 75%, 80%, 85%, 90%, 95%, 96%, 97%, 98%, or 99% sequence identity to SEQ ID NO:1.

4.1.2. 5′ Untranslated Region (5′ UTR)

Expression cassettes of the present disclosure further comprise, operably linked at the 3′ end of the promoter, a sequence that corresponds to a 5′ untranslated region (5′ UTR) in the mRNA resulting from transcription of the expression cassette that is operable in filamentous fungi (for convenience referred to as a “5′ UTR” in the expression cassette). The 5′ UTR can comprise a transcription start site and other features that increase transcription or translation, such as a ribosome binding site.

The 5′ UTR can range in length, from about 50 nucleotides to about 500 nucleotides. In some embodiments, the 5′ UTR is about 50 nucleotides, about 100 nucleotides, about 150 nucleotides, about 200 nucleotides, about 250 nucleotides, about 300 nucleotides, about 350 nucleotides, about 400 nucleotides, about 450 nucleotides, or about 500 nucleotides in length.

The 5′ UTRs for use in the expression cassettes of the present disclosure can be derived from any number of sources, including from a plant gene, a plant virus gene, a yeast gene, a filamentous fungal, gene, or a gene encoding the polypeptide of interest. The 5′ UTR can comprise a nucleotide sequence corresponding to all of a fragment of a 5′UTR from a filamentous fungal gene. The 5′ UTR can comprise a nucleotide sequence corresponding to all or a fragment of the 5′ UTR of a gene encoding a first polypeptide coding sequence of the expression cassette. The 5′ UTR of the expression cassette can be from the same or from a different species as the promoter. In some embodiments, the 5′ UTR is from a different species as the promoter. In some embodiments, the 5′ UTR is not a mammalian 5′ UTR.

The 5′ UTR of the expression cassette can suitably include a nucleotide sequence corresponding to all or a fragment of a 5′ UTR from a filamentous fungal gene. Where the 5′ UTR is derived from a filamentous fungal gene, it may be from a gene native to the filamentous fungal species in which the expression construct is intended to operate. In some embodiments, the 5′ UTR comprises a nucleotide sequence corresponding to all or a fragment of a gene native to an Aspergillus, Trichoderma, Chrysosporium, Cephalosporium, Neurospora, Podospora, Endothia, Cochiobolus, Pyricularia, Rhizomucor, Hansenula, Humicola, Mucor, Tolypocladium, Fusarium, Penicillium, Talaromyces, Emericella, Hypocrea, Acremonium, Aureobasidium, Beauveria, Cephalosporium, Ceriporiopsis, Chaetomium, Paecilomyces, Claviceps, Cryptococcus, Cyathus, Gilocladium, Magnaporthe, Myceliophthora, Myrothecium, Phanerochaete, Paecilomyces, Rhizopus, Schizophylum, Stagonospora, Thermomyces, Thermoascus, Thielavia, Trichophyton, Trametes, or Pleurotus species.

Exemplary filamentous fungal species from which the 5′ UTRs can be derived include Aspergillus awamori, Aspergillus fumigatus, Aspergillus foetidus, Aspergillus japonicus, Aspergillus nidulans, Aspergillus niger, Aspergillus oryzae, Chrysosporium lucknowense, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Neurospora intermedia, Penicillium purpurogenum, Penicillium canescens, Penicillium solitum, Penicillium funiculosum, Phanerochaete chrysosporium, Phlebia radiate, Pleurotus eryngii, Thielavia terrestris, Trichoderma harzianum, Trichoderma longibrachiatum, Trichoderma reesei, and Trichoderma viride.

In a specific embodiment, the 5′ UTR comprises a nucleotide sequence corresponding to all or a fragment of the 5′ UTR from a gene native to Trichoderma reesei, such as the Trichoderma reesei cbh1, cbh2, egl1, egl2, egl5, xln1 and xln2 genes. In exemplary embodiments, the 5′ UTR comprises a nucleotide sequence corresponding to a fragment of the 5′ UTR of the glyceraldehyde-3-phosphate dehydrogenase (gpd) gene of Trichoderma reesei, for example, a 100 nucleotide, 150 nucleotide, or a 200 nucleotide fragment of the Trichoderma reesei gpd gene. In some embodiments, the 5′ UTR of the expression cassette comprises a nucleotide sequence having at least 70%, 75%, 80%, 85%, 90%, 95%, 96%, 97%, 98%, or 99% sequence identity to any one of SEQ ID NO:2, SEQ ID NO:3, or SEQ ID NO:4.

4.1.3. Polypeptide Coding Sequence

The expression cassettes described herein are intended to allow expression of any polypeptide of interest (“POI”) in filamentous fungal cells. As such, the identity of the polypeptide coding sequence is not limited to any particular type of polypeptide or to polypeptides from any particular source. It can be eukaryotic or prokaryotic. The polypeptide coding sequence can be from a gene native to the recombinant filamentous fungal cell into which the expression cassette is intended to be introduced (e.g., from a filamentous fungus such as Trichoderma reesei or Aspergillus niger) or heterologous to the recombinant filamentous fungal cell into which the expression cassette is intended to be introduced (e.g., from a plant, animal, virus, or non-filamentous fungus).

The POI coding sequence can encode an enzyme such as a carbohydrase, such as a liquefying and saccharifying α-amylase, an alkaline α-amylase, a β-amylase, a cellulase; a dextranase, an α-glucosidase, an α-galactosidase, a glucoamylase, a hemicellulase, a pentosanase, a xylanase, an invertase, a lactase, a naringanase, a pectinase or a pullulanase; a protease such as an acid protease, an alkali protease, bromelain, ficin, a neutral protease, papain, pepsin, a peptidase, rennet, rennin, chymosin, subtilisin, thermolysin, an aspartic proteinase, or trypsin; a lipase or esterase, such as a triglyceridase, a phospholipase, acyl transferase, a pregastric esterase, a phosphatase, a phytase, an amidase, an iminoacylase, a glutaminase, a lysozyme, or a penicillin acylase; an isomerase such as glucose isomerase; an oxidoreductases, e.g., an amino acid oxidase, a catalase, a chloroperoxidase, a glucose oxidase, a hydroxysteroid dehydrogenase or a peroxidase; a lyase such as a acetolactate decarboxylase, an aspartic β-decarboxylase, a fumarese or a histadase; a transferase such as cyclodextrin glycosyltransferase; or a ligase, for example.

In particular embodiments, the enzyme is an aminopeptidase, a carboxypeptidase, a chitinase, a cutinase, a deoxyribonuclease, an α-galactosidase, a β-galactosidase, a β-glucosidase, a laccase, a mannosidase, a mutanase, a pectinolytic enzyme, a polyphenoloxidase, ribonuclease or transglutaminase.

In other particular embodiments, the enzyme is an α-amylase, a cellulase; an α-glucosidase, an α-galactosidase, a glucoamylase, a hemicellulase, a xylanase, a pectinase, a pullulanase; an acid protease, an alkali protease, an aspartic proteinase, a lipase, a cutinase or a phytase.

In certain aspects, the POI is a cellulase another protein useful in a cellulotyic reaction, for example a hemicellulase or an accessory polypeptide. Cellulases are known in the art as enzymes that hydrolyze cellulose β-1,4-glucan or β D-glucosidic linkages) resulting in the formation of glucose, cellobiose, cellooligosaccharides, and the like. Cellulase enzymes have been traditionally divided into three major classes: endoglucanases (“EG”), exoglucanases or cellobiohydrolases (EC 3.2.1.91) (“CBH”) and β-glucosidases (EC 3.2.1.21) (“BG”) (Knowles et al., 1987, TIBTECH 5:255-261; Schulein, 1988, Methods in Enzymology 160(25):234-243). Accessory proteins

Endoglucanases:

Endoglucanases break internal bonds and disrupt the crystalline structure of cellulose, exposing individual cellulose polysaccharide chains (“glucans”). Endoglucanases include polypeptides classified as Enzyme Commission no. (“EC”) 3.2.1.4) or which are capable of catalyzing the endohydrolysis of 1,4-β-D-glucosidic linkages in cellulose, lichenin or cereal β-D-glucans. Enzyme Commission numbering is a numerical classification scheme for enzymes.

Examples of suitable bacterial endoglucanases include, but are not limited to, Acidothermus cellulolyticus endoglucanase (WO 91/05039; WO 93/15186; U.S. Pat. No. 5,275,944; WO 96/02551; U.S. Pat. No. 5,536,655, WO 00/70031, WO 05/093050); Thermobifida fusca endoglucanase 111 (WO 05/093050); and Thermobifida fusca endoglucanase V (WO 05/093050).

Examples of suitable fungal endoglucanases include, but are not limited to, Trichoderma reesei endoglucanase I (Penttila et al., 1986, Gene 45: 253-263; GenBank accession no. M15665); Trichoderma reesei endoglucanase II (Saloheimo et al., 1988, Gene 63:11-22; GenBank accession no. M19373); Trichoderma reesei endoglucanase 111 (Okada et al., 1988, Appl. Environ. Microbiol. 64: 555-563; GenBank accession no. AB003694); Trichoderma reesei endoglucanase IV (Saloheimo et al., 1997, Eur. J. Biochem. 249: 584-591; GenBank accession no. Y11113); and Trichoderma reesei endoglucanase V (Saloheimo et al., 1994, Molecular Microbiology 13: 219-228; GenBank accession no. Z33381); Aspergillus aculeatus endoglucanase (Ooi et al., 1990, Nucleic Acids Research 18: 5884); Aspergillis kawachii endoglucanase (Sakamoto et al., 1995, Current Genetics 27: 435-439); Chrysosporium sp. C1 endoglucanase (U.S. Pat. No. 6,573,086; GenPept accession no. AAQ38150); Corynascus heterothallicus endoglucanase (U.S. Pat. No. 6,855,531; GenPept accession no. AAY00844); Erwinia carotovara endoglucanase (Saarilahti et al., 1990, Gene 90: 9-14); Fusarium oxysporum endoglucanase (GenBank accession no. L29381); Humicola grisea var. thermoidea endoglucanase (GenBank accession no. AB003107); Melanocarpus albomyces endoglucanase (GenBank accession no. MAL515703); Neurospora crassa endoglucanase (GenBank accession no. XM.sub.—324477); Piromyces equi endoglucanase (Eberhardt et al., 2000, Microbiology 146: 1999-2008; GenPept accession no. CAB92325); Rhizopus oryzae endoglucanase (Moriya et al., 2003, J. Bacteriology 185: 1749-1756; GenBank accession nos. AB047927, AB056667, and AB056668); and Thielavia terrestris endoglucanase (WO 2004/053039; EMBL accession no. CQ827970).

Cellobiohydrolases:

Cellobiohydrolases incrementally shorten the glucan molecules, releasing mainly cellobiose units (a water-soluble β-1,4-linked dimer of glucose) as well as glucose, cellotriose, and cellotetraose. Cellobiohydrolases include polypeptides classified as EC 3.2.1.91 or which are capable of catalyzing the hydrolysis of 1,4-β-D-glucosidic linkages in cellulose or cellotetraose, releasing cellobiose from the ends of the chains. Exemplary cellobiohydrolases include Trichoderma reesei cellobiohydrolase I (CEL7A) (Shoemaker et al., 1983, Biotechnology (N.Y.) 1: 691-696); Trichoderma reesei cellobiohydrolase II (CEL6A) (Teeri et al., 1987, Gene 51: 43-52); Chrysosporium lucknowense CEL7 cellobiohydrolase (WO 2001/79507); Myceliophthora thermophila CEL7 (WO 2003/000941); and Thielavia terrestris cellobiohydrolase (WO 2006/074435).

β-Glucosidases:

β-Glucosidases split cellobiose into glucose monomers. β-glucosidases include polypeptides classified as EC 3.2.1.21 or which are capable of catalyzing the hydrolysis of terminal, non-reducing β-D-glucose residues with release of β-D-glucose. Exemplary β-glucosidases can be obtained from Cochliobolus heterostrophus (SEQ ID NO:34), Aspergillus oryzae (WO 2002/095014), Aspergillus fumigatus (WO 2005/047499), Penicillium brasilianum (e.g., Penicillium brasilianum strain IBT 20888) (WO 2007/019442), Aspergillus niger (Dan et al., 2000, J. Biol. Chem. 275: 4973-4980), Aspergillus aculeatus (Kawaguchi et al., 1996, Gene 173: 287-288), Penicilium funiculosum (WO 2004/078919), S. pombe (Wood et al., 2002, Nature 415: 871-880), T reesei (e.g., β-glucosidase 1 (U.S. Pat. No. 6,022,725), β-glucosidase 3 (U.S. Pat. No. 6,982,159), β-glucosidase 4 (U.S. Pat. No. 7,045,332), β-glucosidase 5 (U.S. Pat. No. 7,005,289), β-glucosidase 6 (U.S. Publication No. 20060258554), or β-glucosidase 7 (U.S. Publication No. 20060258554)).

Hemicellulases: A POI can be any class of hemicellulase, including an endoxylanase, a β-xylosidase, an α-L-arabionofuranosidase, an α-D-glucuronidase, an acetyl xylan esterase, a feruloyl esterase, a coumaroyl esterase, an α-galactosidase, a α-galactosidase, a β-mannanase or a β-mannosidase.

Endoxylanases suitable as POIs include any polypeptide classified EC 3.2.1.8 or which is capable of catalyzing the endohydrolysis of 1,4-β-D-xylosidic linkages in xylans. Endoxylanases also include polypeptides classified as EC 3.2.1.136 or which are capable of hydrolyzing 1,4 xylosidic linkages in glucuronoarabinoxylans.

β-xylosidases include any polypeptide classified as EC 3.2.1.37 or which is capable of catalyzing the hydrolysis of 1,4-β-D-xylans to remove successive D-xylose residues from the non-reducing termini β-xylosidases may also hydrolyze xylobiose.

α-L-arabinofuranosidases include any polypeptide classified as EC 3.2.1.55 or which is capable of acting on α-L-arabinofuranosides, α-L-arabinans containing (1,2) and/or (1,3)- and/or (1,5)-linkages, arabinoxylans or arabinogalactans.

α-D-glucuronidases include any polypeptide classified as EC 3.2.1.139 or which is capable of catalyzing a reaction of the following form: α-D-glucuronoside+H(2)O=an alcohol+D-glucuronate. α-D-glucuronidases may also hydrolyse 4-O-methylated glucoronic acid, which can also be present as a substituent in xylans. α-D-glucuronidases also include polypeptides classified as EC 3.2.1.131 or which are capable of catalying the hydrolysis of α-1,2-(4-O-methyl)glucuronosyl links.

Acetyl xylan esterases include any polypeptide classified as EC 3.1.1.72 or which is capable of catalyzing the deacetylation of xylans and xylo-oligosaccharides. Acetyl xylan esterases may catalyze the hydrolysis of acetyl groups from polymeric xylan, acetylated xylose, acetylated glucose, α-napthyl acetate or p-nitrophenyl acetate but, typically, not from triacetylglycerol. Acetyl xylan esterases typically do not act on acetylated mannan or pectin.

Feruloyl esterases include any polypeptide classified as EC 3.1.1.73 or which is capable of catalyzing a reaction of the form: feruloyl-saccharide+H(2)O=ferulate+saccharide. The saccharide may be, for example, an oligosaccharide or a polysaccharide. A feruloyl esterase may catalyze the hydrolysis of the 4-hydroxy-3-methoxycinnamoyl (feruloyl) group from an esterified sugar, which is usually arabinose in natural substrates, while p-nitrophenol acetate and methyl ferulate are typically poorer substrates. Feruloyl esterase are sometimes considered hemicellulase accessory enzymes, since they may help xylanases and pectinases to break down plant cell wall hemicellulose and pectin.

Coumaroyl esterases include any polypeptide classified as EC 3.1.1.73 or which is capable of catalyzing a reaction of the form: coumaroyl-saccharide+H(2)O=coumarate+saccharide. The saccharide may be, for example, an oligosaccharide or a polysaccharide. Because some coumaroyl esterases are classified as EC 3.1.1.73 they may also be referred to as feruloyl esterases.

α-galactosidases include any polypeptide classified as EC 3.2.1.22 or which is capable of catalyzing the hydrolysis of terminal, non-reducing α-D-galactose residues in α-D-galactosides, including galactose oligosaccharides, galactomannans, galactans and arabinogalactans. α-galactosidases may also be capable of hydrolyzing α-D-fucosides.

β-galactosidases include any polypeptide classified as EC 3.2.1.23 or which is capable of catalyzing the hydrolysis of terminal non-reducing β-D-galactose residues in β-D-galactosides. β-galactosidases may also be capable of hydrolyzing α-L-arabinosides.

β-mannanases include any polypeptide classified as EC 3.2.1.78 or which is capable of catalyzing the random hydrolysis of 1,4-β-D-mannosidic linkages in mannans, galactomannans and glucomannans.

β-mannosidases include any polypeptide classified as EC 3.2.1.25 or which is capable of catalyzing the hydrolysis of terminal, non-reducing β-D-mannose residues in β-D-mannosides.

Suitable hemicellulases include T reesei α-arabinofuranosidase I (ABF1), α-arabinofuranosidase I1 (ABF2), α-arabinofuranosidase III (ABF3), α-galactosidase I (AGL1), α-galactosidase I1 (AGL2), α-galactosidase III (AGL3), acetyl xylan esterase I (AXE1), acetyl xylan esterase III (AXE3), endoglucanase V1 (EG6), endoglucanase VIII (EG8), α-glucuronidase I (GLR1), β-mannanase (MAN1), polygalacturonase (PEC2), xylanase I (XYN1), xylanase I1 (XYN2), xylanase 111 (XYN3), and β-xylosidase (BXL1).

Accessory Polypeptides:

Accessory polypeptides are present in cellulase preparations that aid in the enzymatic digestion of cellulose (see, e.g., WO 2009/026722 and Harris et al., 2010, Biochemistry, 49:3305-3316). In some embodiments, the accessory polypeptide is an expansin or swollenin-like protein. Expansins are implicated in loosening of the cell wall structure during plant cell growth (see, e.g., Salheimo et al., 2002, Eur. J. Biochem., 269:4202-4211). Expansins have been proposed to disrupt hydrogen bonding between cellulose and other cell wall polysaccharides without having hydrolytic activity. In this way, they are thought to allow the sliding of cellulose fibers and enlargement of the cell wall. Swollenin, an expansin-like protein, contains an N-terminal Carbohydrate Binding Module Family 1 domain (CBD) and a C-terminal expansin-like domain. In some embodiments, an expansin-like protein and/or swollenin-like protein comprises one or both of such domains and/or disrupts the structure of cell walls (e.g., disrupting cellulose structure), optionally without producing detectable amounts of reducing sugars. Other types of accessory proteins include cellulose integrating proteins, scaffoldins and/or a scaffoldin-like proteins (e.g., CipA or CipC from Clostridium thermocellum or Clostridium cellulolyticum respectively). Other exemplary accessory proteins are cellulose induced proteins and/or modulating proteins (e.g., as encoded by cip1 or cip2 gene and/or similar genes from Trichoderma reesei; see e.g., Foreman et al., 2003, J. Biol. Chem., 278:31988-31997.

The POI coding sequence of an expression cassette of the disclosure can also encode a therapeutic polypeptide (i.e., a polypeptide having a therapeutic biological activity). Examples of suitable therapeutic polypeptides include: erythropoietin, cytokines such as interferon-α, interferon-β, interferon-γ, interferon-o, and granulocyte-CSF, GM-CSF, coagulation factors such as factor VIII, factor IX, and human protein C, antithrombin III, thrombin, soluble IgE receptor α-chain, IgG, IgG fragments, IgG fusions, IgM, IgA, interleukins, urokinase, chymase, and urea trypsin inhibitor, IGF-binding protein, epidermal growth factor, growth hormone-releasing factor, annexin V fusion protein, angiostatin, vascular endothelial growth factor-2, myeloid progenitor inhibitory factor-1, osteoprotegerin, α-1-antitrypsin, α-feto proteins, DNase II, kringle 3 of human plasminogen, glucocerebrosidase, TNF binding protein 1, follicle stimulating hormone, cytotoxic T lymphocyte associated antigen 4-Ig, transmembrane activator and calcium modulator and cyclophilin ligand, soluble TNF receptor Fc fusion, glucagon like protein 1 and IL-2 receptor agonist. Antibodies, e.g., monoclonal antibodies (including but not limited to chimeric and humanized antibodies), are of particular interest.

In a further embodiment, the POI coding sequence can encode a reporter polypeptide. Such reporter polypeptides may be optically detectable or colorigenic, for example. In this embodiment, the polypeptide may be a β-galactosidase (lacZ), β-glucuronidase (GUS), luciferase, alkaline phosphatase, nopaline synthase (NOS), chloramphenicol acetyltransferase (CAT), horseradish peroxidase (HRP) or a fluorescent protein green, e.g., green fluorescent protein (GFP), or a derivative thereof.

Where the POI coding sequence is from a eukaryotic gene, the polypeptide coding sequence can, but need not, include introns which can be spliced out during post-transcriptional processing of the transcript in the cell.

For some applications, it may be desirable for the polypeptide produced to be secreted by the filamentous fungal cell. For such application, the POI coding sequence can include, or be engineered to include, a signal sequence encoding a leader peptide that directs the POI to the filamentous fungal cell's secretory pathway. The signal sequence, when present, is in an appropriate translation reading frame with the mature POI coding sequence. Accordingly, the POI coding sequence can further encode a signal sequence operably linked to the N-terminus of the POI, where the signal sequence contains a sequence of amino acids that directs the POI to the secretory system of the recombinant filamentous fungal cell, resulting in secretion of the mature POI from the recombinant filamentous fungal cell into the medium in which the recombinant filamentous fungal cell is growing. The signal sequence is cleaved from the fusion protein prior to secretion of the mature POI. The signal sequence employed can be endogenous or non-endogenous to the POI and/or the recombinant filamentous fungal cell. Preferably, the signal sequence is a signal sequence that facilitates protein secretion from a filamentous fungal (e.g., Trichoderma or Aspergillus) cell and can be the signal sequence of a protein that is known to be highly secreted from filamentous fungi. Such signal sequences include, but are not limited to: the signal sequence of cellobiohydrolase I, cellobiohydrolase II, endoglucanase I, endoglucanase II, endoglucanase III, α-amylase, aspartyl proteases, glucoamylase, mannanase, glycosidase and barley endopeptidase B (see Saarelainen, 1997, Appl. Environ. Microbiol. 63:4938-4940), for example. Specific examples include the signal sequence from Aspergillus oryzae TAKA α-amylase, Aspergillus niger neutral α-amylase, Aspergillus niger glucoamylase, Rhizomucor miehei aspartic proteinase, Humicola insolens cellulase, and Humicola lanuginosa lipase. Other examples of signal sequences are those originating from the α-factor gene of a yeast (e.g., Saccharomyces, Kluyveromyces and Hansenula) or a Bacillus α-amylase. In certain embodiments, therefore, the POI coding sequence includes a sequence encoding a signal sequence, yielding a POI in the form of a polypeptide comprising an N-terminal signal sequence for secretion of the protein from the recombinant filamentous fungal cell.

In certain embodiments, the POI coding sequence can encode a fusion protein. In addition to POIs comprising signal sequences as described above, the fusion protein can further contain a “carrier protein,” which is a portion of a protein that is endogenous to and highly secreted by the filamentous fungal cell. Suitable carrier proteins include those of Trichoderma reesei mannanase I (Man5A, or MANI), Trichoderma reesei cellobiohydrolase II (Cel6A, or CBHII) (see, e.g., Paloheimo et al., 2003, Appl. Environ. Microbiol. 69(12): 7073-7082) or Trichoderma reesei cellobiohydrolase I (CBHI). In one embodiment, the carrier protein is a truncated Trichoderma reesei CBHI protein that includes the CBHI core region and part of the CBHI linker region. An expression cassette of the disclosure can therefore include a coding sequence for a fusion protein containing, from the N-terminus to C-terminus, a signal sequence, a carrier protein and a POI in operable linkage.

In certain embodiments, the POI coding sequence can be codon optimized for expression of the protein in a particular filamentous fungal cell. Since codon usage tables listing the usage of each codon in many cells are known in the art (see, e.g., Nakamura et al., 2000, Nucl. Acids Res. 28:292) or readily derivable, such coding sequence can be readily designed.

The expression cassettes described herein comprise at least a first polypeptide coding sequence encoding a first polypeptide, but may optionally comprise second, third, fourth, etc. polypeptide coding sequences encoding second, third, fourth, etc. polypeptides.

4.1.4. 3′ Untranslated Region (3′ UTR)

Expression cassettes of the present disclosure further comprise, operably linked at the 3′ end of the first, and any optional additional, polypeptide coding sequence, a sequence that corresponds to a 3′ untranslated region (3′ UTR) in the mRNA resulting from transcription of the expression cassette (for convenience referred to as a “3′ UTR” in the expression cassette). The 3′ UTR of the expression cassette comprises at least a polyadenylation signal, directing cleavage and polyadenylation of the transcript. The 3′ UTR can optionally comprise other features important for nuclear export, translation, and/or stability of the mRNA, such as for example, a termination signal.

The 3′ UTR can range in length from about 50 nucleotides to about 2000 or nucleotides or longer. In some embodiments, the 5′ UTR is about 50 nucleotides, about 100 nucleotides, about 150 nucleotides, about 200 nucleotides, about 250 nucleotides, about 300 nucleotides, about 350 nucleotides, about 400 nucleotides, about 450 nucleotides, about 500 nucleotides, about 600 nucleotides, about 700 nucleotides, about 800 nucleotides, about 900 nucleotides, about 1000 nucleotides, or about 2000 nucleotides in length or more.

Suitable 3′ UTRs for use in the expression cassettes of the present disclosure can be derived from any number of sources, including from a plant gene, a plant virus gene, a yeast gene, a filamentous fungal, gene, or a gene encoding the polypeptide of interest. The 3′ UTR can comprise a nucleotide sequence corresponding to all or a fragment of a 3′UTR from a plant gene, a plant viral gene, a yeast gene or a filamentous fungal gene. The 3′ UTR can comprise a nucleotide sequence corresponding to all or a fragment of the 3′ UTR of a gene encoding a first, second, or further polypeptide coding sequence of the expression cassette. The 3′ UTR can be from the same or a different species as one other component in the expression cassette (e.g., the promoter or the polypeptide coding sequence). The 3′ UTR can be from the same species as the filamentous fungal cell in which the expression construct is intended to operate.

The 3′ UTR of an expression cassette of the disclosure may also suitably be derived from a plant gene or a plant viral gene, for example a gene native to a virus belonging to one of the Caulimoviridae, Geminiviridae, Reoviridae, Rhabdoviridae, Virgaviridae, Alphaflexiviridae, Potyviridae, Betaflexiviridae, Closteroviridae, Tymoviridae, Luteoviridae, Tombusviridae, Sobemoviruses, Neopviruses, Secoviridae and Bromoviridae families. In some embodiments, the 3′ UTR comprises a nucleotide sequence corresponding to all or a fragment of a 3′ UTR from a Caulimoviridae virus. In specific embodiments, the 3′ UTR comprises a nucleotide sequence corresponding to all or a fragment of a CaMV 35S transcript 3′UTR.

The 3′ UTR of an expression cassette of the disclosure may also suitably be derived from a mammalian gene or a mammalian viral gene, for example a gene native to a virus belonging to one of the viruses belong to one of the Retroviridae, Picornaviridae, Calciviridae, Togaviridae, Flaviridae, Coronaviridae, Rhabdoviridae, Filoviridae, Paramyxoviridae, Orthomyxoviridae, Bungaviridae, Arenaviridae, Reoviridae, Birnaviridae, Hepadnaviridae, Parvoviridae, Papovaviridae, Adenoviridae, Herpesviridae, Polyomaviridae, Poxyiridae and Iridoviridae families.

The 3′ UTR of an expression cassette of the disclosure may also suitably be derived from a filamentous fungal gene. Where the 3′ UTR is derived from a filamentous fungal gene, it may be from a gene native to the filamentous fungal species in which the expression construct is intended to operate. Exemplary filamental fungal species the 3′ UTR comprises a nucleotide sequence corresponding to all or a fragment of a gene native to a Aspergillus, Trichoderma, Chrysosporium, Cephalosporium, Neurospora, Podospora, Endothia, Cochiobolus, Pyricularia, Rhizomucor, Hansenula, Humicola, Mucor, Tolypocladium, Fusarium, Penicillium, Talaromyces, Emericella, Hypocrea, Acremonium, Aureobasidium, Beauveria, Cephalosporium, Ceriporiopsis, Chaetomium, Paecilomyces, Claviceps, Cryptococcus, Cyathus, Gilocladium, Magnaporthe, Myceliophthora, Myrothecium, Phanerochaete, Paecilomyces, Rhizopus, Schizophylum, Stagonospora, Thermomyces, Thermoascus, Thielavia, Trichophyton, Trametes, and Pleurotus species.

Species of filamentous fungi from which the 3′ UTR can be derived include Aspergillus awamori, Aspergillus fumigatus, Aspergillus foetidus, Aspergillus japonicus, Aspergillus nidulans, Aspergillus niger, Aspergillus oryzae, Chrysosporium lucknowense, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Neurospora intermedia, Penicillium purpurogenum, Penicillium canescens, Penicillium solitum, Penicillium funiculosum, Phanerochaete chrysosporium, Phlebia radiate, Pleurotus eryngii, Thielavia terrestris, Trichoderma harzianum, Trichoderma longibrachiatum, Trichoderma reesei, and Trichoderma viride.

In a specific embodiment, the 3′ UTR comprises a nucleotide sequence corresponding to all or a fragment of the 3′ UTR from a gene native to Trichoderma reesei, such as the Trichoderma reesei CBHI, cbh2, egl1, egl2, egl5, xln1 and xln2 genes. In an exemplary embodiment, the 3′ UTR comprises a nucleotide sequence corresponding to a fragment of the 3′ UTR of the glyceraldehyde-3-phosphate dehydrogenase (gpd) gene of Trichoderma reesei. In another exemplary embodiment, the 3′ UTR comprises the nucleotide sequence of all or a fragment of the 3′ UTR of a gene encoding CBHI.

In other exemplary embodiments, the 3′ UTR comprises a nucleotide sequence corresponding to all or a fragment of the 3′UTR from an Aspergillus niger or Aspergillus awamori glucoamylase gene (Nunberg et al., 1984, Mol. Cell. Biol. 4:2306-2315 and Boel et al., 1984, EMBO Journal, 3:1097-1102), an Aspergillus nidulans anthranilate synthase gene, an Aspergillus oryzae TAKA amylase gene, or the Aspergillus nidulans trpc gene (Punt et al., 1987, Gene 56:117-124).

In yet other exemplary embodiments, the 3′ UTR comprises the nucleotide sequence corresponding to all or a fragment of a 3′ UTR from a Cochliobolus species, e.g., Cochliobolus heterostrophus. In a specific embodiment, the 3′ UTR comprises the nucleotide sequence of all or a fragment of the 3′ UTR of a Cochliobolus heterostrophus gene encoding β-glucosidase.

In a specific embodiment, the 3′ UTR comprises the nucleotide sequence of SEQ ID NO:5. Suitable 3′ UTRs can comprise a nucleotide sequence having at least 70%, 75%, 80%, 85%, 90%, 95%, 96%, 97%, 98%, or 99% sequence identity to SEQ ID NO:5.

4.2. Methods Of Making Expression Cassettes

Techniques for the manipulation of nucleic acids, including techniques for the synthesis, isolation, cloning, detection, and identification are well known in the art and are well described in the scientific and patent literature. See, e.g., Sambrook et al., eds., Molecular Cloning: A Laboratory Manual (2nd Ed.), Vols. 1-3, Cold Spring Harbor Laboratory (1989); Ausubel et al., eds., Current Protocols in Molecular Biology, John Wiley & Sons, Inc., New York (1997); Tijssen, ed., Laboratory Techniques in Biochemistry and Molecular Biology Hybridization With Nucleic Acid Probes, Part I. Theory and Nucleic Acid Preparation, Elsevier, N.Y. (1993). Nucleic acids comprising the expression cassettes described herein or components thereof include isolated, synthetic, and recombinant nucleic acids.

Expression cassettes and components thereof can readily be made and manipulated from a variety of sources, either by cloning from genomic or complementary DNA, e.g., by using the well known polymerase chain reaction (PCR). See, for example, Innis et al., 1990, PCR Protocols: A Guide to Methods and Application, Academic Press, New York. Expression cassettes and components thereof can also be made by chemical synthesis, as described in, e.g., Adams, 1983, J. Am. Chem. Soc. 105:661; Belousov, 1997, Nucleic Acids Res. 25:3440-3444; Frenkel, 1995, Free Radic. Biol. Med. 19:373-380; Blommers, 1994, Biochemistry 33:7886-7896; Narang, 1979, Meth. Enzymol. 68:90; Brown, 1979, Meth. Enzymol. 68:109; Beaucage, 1981, Tetra. Lett. 22:1859; U.S. Pat. No. 4,458,066.

The promoter, 5′ UTR and 3′ UTR of an expression cassette of the disclosure be operably linked in a vector. The vector can also include the POI coding sequence, or one or more convenient restriction sites between the 5′ UTR and 3′ UTR sequences to allow for insertion or substitution of the POI coding sequence. The procedures used to ligate the components described herein to construct the recombinant expression vectors are well known to one skilled in the art (see, e.g., Sambrook et al., eds., Molecular Cloning: A Laboratory Manual (2nd Ed.), Vols. 1-3, Cold Spring Harbor Laboratory (1989)). As will be described further below, vectors comprising expression cassettes described herein typically contain features making them suitable for introduction into filamentous fungal cells.

4.3. Recombinant Filamentous Fungal Cells

The expression cassettes described herein are usefully expressed in filamentous fungal cells suited to the production of one or more polypeptides of interest. Accordingly, the present disclosure provides recombinant filamentous fungal cells comprising expression cassettes of the disclosure and methods of introducing expression cassettes into filamentous fungal cells.

Suitable filamentous fungal cells include all filamentous forms of the subdivision Eumycotina (see, Alexopoulos, C. J. (1962), INTRODUCTORY MYCOLOGY, Wiley, New York). These fungi are characterized by a vegetative mycelium with a cell wall composed of chitin, cellulose, and other complex polysaccharides. The filamentous fungal cell can be from a fungus belonging to any species of Aspergillus, Trichoderma, Chrysosporium, Cephalosporium, Neurospora, Podospora, Endothia, Cochiobolus, Pyricularia, Rhizomucor, Hansenula, Humicola, Mucor, Tolypocladium, Fusarium, Penicillium, Talaromyces, Emericella, Hypocrea, Acremonium, Aureobasidium, Beauveria, Cephalosporium, Ceriporiopsis, Chaetomium, Paecilomyces, Claviceps, Cryptococcus, Cyathus, Gilocladium, Magnaporthe, Myceliophthora, Myrothecium, Phanerochaete, Paecilomyces, Rhizopus, Schizophylum, Stagonospora, Thermomyces, Thermoascus, Thielavia, Trichophyton, Trametes, and Pleurotus. More preferably, the recombinant cell is a Trichoderma sp. (e.g., Trichoderma reesei), Penicillium sp., Humicola sp. (e.g., Humicola insolens); Aspergillus sp. (e.g., Aspergillus niger), Chrysosporium sp., Fusarium sp., or Hypocrea sp. Suitable cells can also include cells of various anamorph and teleomorph forms of these filamentous fungal genera.

Exemplary filamentous fungal species include but are not limited to Aspergillus awamori, Aspergillus fumigatus, Aspergillus foetidus, Aspergillus japonicus, Aspergillus nidulans, Aspergillus niger, Aspergillus oryzae, Chrysosporium lucknowense, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Neurospora intermedia, Penicillium purpurogenum, Penicillium canescens, Penicillium solitum, Penicillium funiculosum, Phanerochaete chrysosporium, Phlebia radiate, Pleurotus eryngii, Thielavia terrestris, Trichoderma harzianum, Trichoderma longibrachiatum, Trichoderma reesei, or Trichoderma viride.

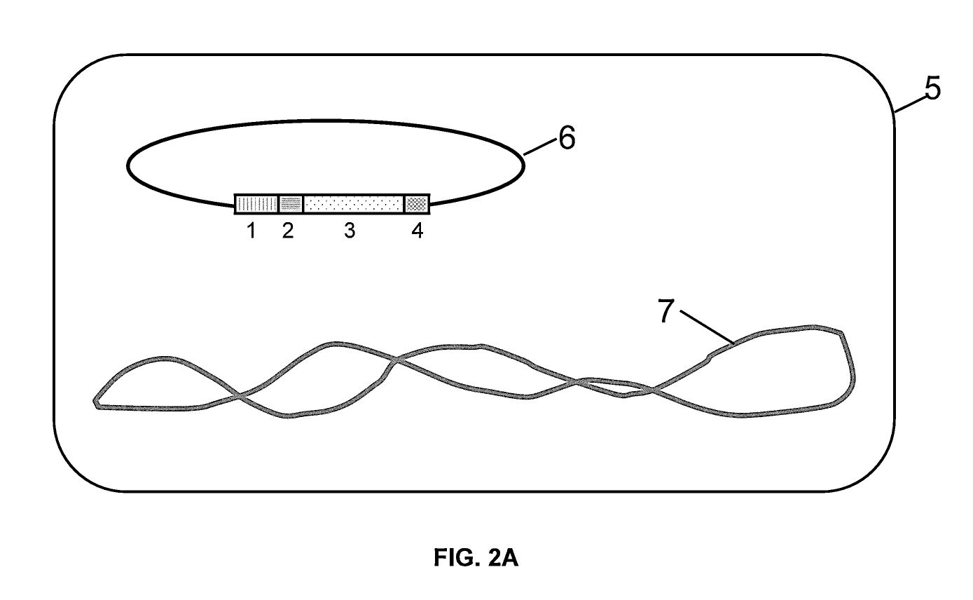

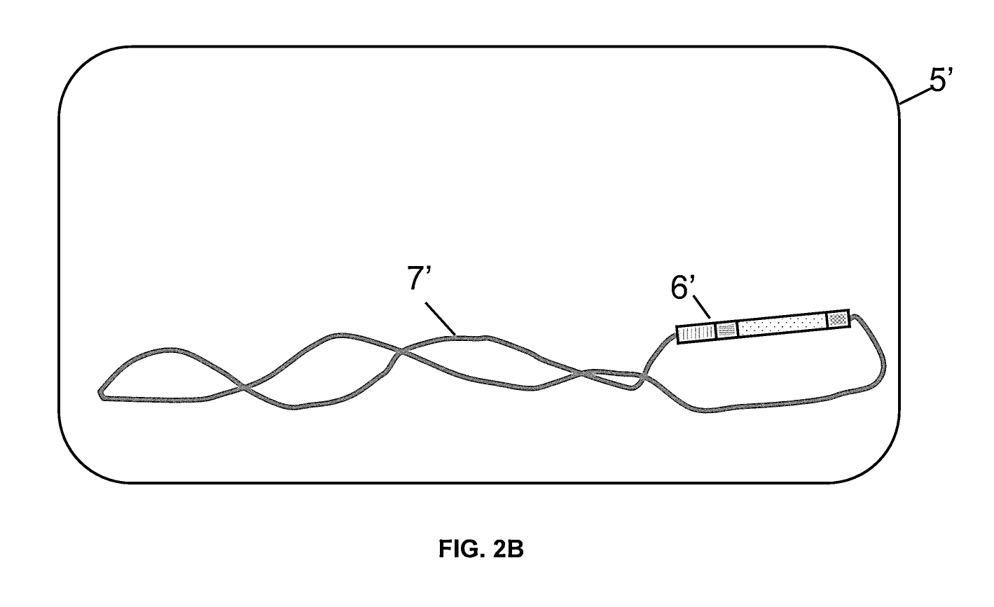

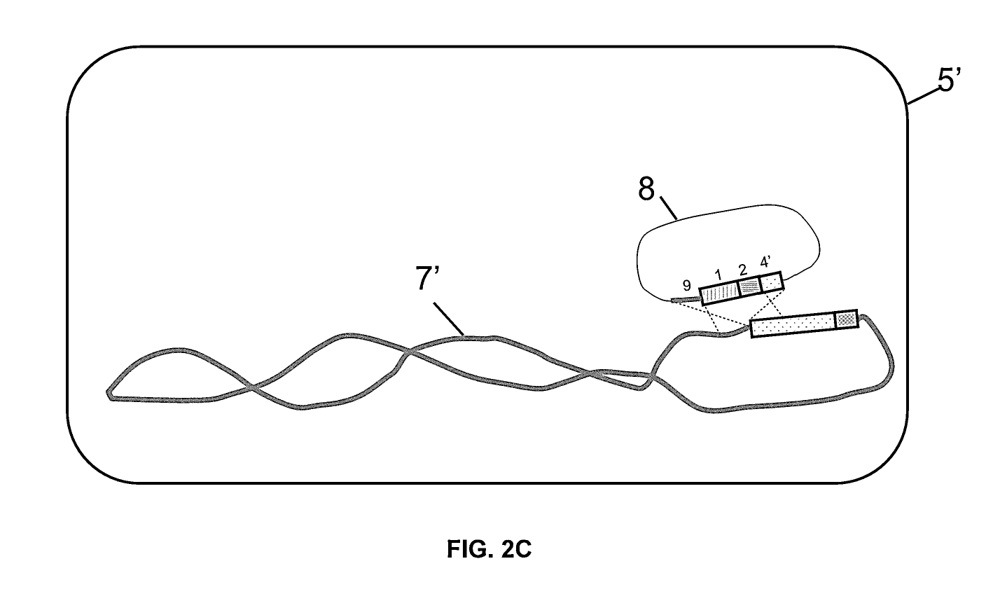

Recombinant filamentous fungal cells comprise an expression cassette as described above in Section 4.1. The expression cassette can be extra-genomic or integrated into the host's genome. FIG. 2A provides a schematic of a recombinant filamentous fungal cell containing an extra-genomic expression cassette. As depicted, the recombinant filamentous fungal cell (5) carrying a vector comprising an expression cassette (6), the expression cassette comprising a promoter (1), a 5′ UTR (2), a polypeptide coding sequence (3), and a 3′ UTR (4). The expression cassette is not integrated into the chromosome (7) of the recombinant filamentous fungal cell (5). FIG. 2B provides a schematic of a recombinant filamentous fungal cell containing a genomic expression cassette. As depicted the recombinant filamentous fungal cell (5′) comprises an expression cassette (6′), which is integrated into the chromosome (7′) of the recombinant filamentous fungal cell (5′).

The recombinant filamentous fungal cell of FIG. 2B can be generated by introducing and integrating a complete expression cassette into the host chromosome. Alternatively, the recombinant filamentous fungal cell of FIG. 2B may be generated by introducing subset of the components of the expression cassette into the chromosome in such a way and in a location so as to recapitulate a complete expression cassette within the host chromosome. For example, as depicted in FIG. 2C, a vector (8) comprising a promoter (1), a 5′ UTR (2), a sequence of a polypeptide coding region homologous to that of a native fungal cell gene (4′), and a sequence homologous to from a region upstream of the native fungal cell gene (9), can be integrated by homologous recombination at a location upstream (on the 5′ end) of the native gene comprising a 3′ UTR in the chromosome (7′) of a filamentous fungal cell to generate a complete expression cassette as depicted in FIG. 2B. In another example, a suitable promoter may be integrated upstream of the 5′ UTR of a native gene in the chromosome. Other combinations are also possible, provided that a genomic expression cassette comprising all four components in the results.

Suitable methods for introducing expression cassettes, as well as methods for integrating expression cassettes into the filamentous fungal cell genome are described in further detail below.

4.4. Vectors

The filamentous fungal cells of the present disclosure are engineered to comprise an expression cassette, resulting in recombinant or engineered filamentous fungal cells. Expression cassettes, or components thereof, can be introduced into filamentous fungal cells by way of suitable vectors. The choice of the vector will typically depend on the compatibility of the vector with the into which the vector is to be introduced (e.g., a filamentous fungal cell or a host cell, such as a bacterial cell, useful for propagating or amplifying the vector), whether autonomous replication of the vector inside the filamentous fungal cell and/or integration of the vector into the filamentous fungal cell genome is desired. The vector can be a viral vector, a phage, a phagemid, a cosmid, a fosmid, a bacteriophage, an artificial chromosome, a cloning vector, an expression vector, a shuttle vector, a plasmid (linear or closed circular), or the like. Vectors can include chromosomal, non-chromosomal and synthetic DNA sequences. Large numbers of suitable vectors are known to those of skill in the art, and are commercially available. Low copy number or high copy number vectors may be employed. Examples of suitable expression and integration vectors are provided in Sambrook et al., eds., Molecular Cloning: A Laboratory Manual (2nd Ed.), Vols. 1-3, Cold Spring Harbor Laboratory (1989), and Ausubel et al., eds., Current Protocols in Molecular Biology, John Wiley & Sons, Inc., New York (1997), and van den Hondel et al. (1991) in Bennett and Lasure (Eds.) MORE GENE MANIPULATIONS IN FUNGI, Academic Press pp. 396-428 and U.S. Pat. No. 5,874,276. Reference is also made to the Filamentous Fungal Genetics Stock Center Catalogue of Strains (FGSC, <www.fgsc.net>) for a list of vectors. Particularly useful vectors include vectors obtained from commercial sources, such as Invitrogen and Promega. Specific vectors suitable for use in filamentous fungal cells include vectors such as pFB6, pBR322, pUC18, pUC100, pDON™201, pDONR™221, pENTR™, pGEM®3Z and pGEM®4Z.

For some applications, it may be desirable for the expression cassette, or components thereof, to be maintained as extra-genomic elements. For such applications, suitable vectors comprising an expression cassette or components are preferably capable of autonomously replicating in a cell, independent of chromosomal replication. Accordingly, in some embodiments, the vector comprises an origin of replication enabling it to replicate autonomously in a cell, such as in a filamentous fungal cell.

For many applications, it is desirable to have a tool for selecting recombinant cells containing the vector. Thus, in some embodiments, the vector comprises a selectable marker. A selectable marker is a gene the product of which provides a selectable trait, e.g., antibiotic, biocide or viral resistance, resistance to heavy metals, or prototrophy in auxotrophs. Selectable markers useful in vectors for transformation of various filamentous fungal strains are known in the art. See, e.g., Finkelstein, chapter 6 in BIOTECHNOLOGY OF FILAMENTOUS FUNGI, Finkelstein et al. Eds. Butterworth-Heinemann, Boston, Mass. (1992), Chap. 6; and Kinghorn et al. (1992) APPLIED MOLECULAR GENETICS OF FILAMENTOUS FUNGI, Blackie Academic and Professional, Chapman and Hall, London). Examples of selectable markers which confer antimicrobial resistance include hygromycin and phleomycin. Further exemplary selectable markers include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), pyr4 (orotidine-5′-monophosphate decarboxylase) and trpC (anthranilate synthase). As a specific example, the amdS gene, allows transformed cells to grow on acetamide as a nitrogen source. See, e.g., Kelley et al., 1985, EMBO J. 4:475-479 and Penttila et al., 1987, Gene 61:155-164. Other specific examples of selectable markers include amdS and pyrG genes of Aspergillus nidulans or Aspergillus oryzae and the bar gene of Streptomyces hygroscopicus.

4.5. Methods of Making Recombinant Filamentous Fungal Cells

Recombinant fungal cells as provided herein, are generated by introducing one or more components of an expression cassette into a suitable filamentous fungal cell. Numerous techniques for introducing nucleic acids into cells, including filamentous fungal cells are known. Nucleic acids may be introduced into the cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation (Davis, L., Dibner, M., Battey, I., Basic Methods in Molecular Biology, (1986)). General transformation techniques are known in the art (See, e.g., Ausubel et al., eds., Current Protocols in Molecular Biology, John Wiley & Sons, Inc., New York (1997); and Sambrook et al., eds., Molecular Cloning: A Laboratory Manual (2nd Ed.), Vols. 1-3, Cold Spring Harbor Laboratory (1989), and Campbell et al., 1989, Curr. Genet. 16:53-56).

Suitable procedures for transformation of various filamentous fungal strains have been described. See e.g., EP 238 023 and Yelton et al, 1984, Proceedings of the National Academy of Sciences USA 81: 1470-1474 for descriptions of transformation in Aspergillus host strains. Reference is also made to Cao et al., 2000, Sci. 9:991-1001 and EP 238 023 for transformation of Aspergillus strains and WO96/00787 for transformation of Fusarium strains. See also, U.S. Pat. No. 6,022,725; U.S. Pat. No. 6,268,328; Harkki et al., 1991, Enzyme Microb. Technol. 13:227-233; Harkki et al., 1989, Bio Technol. 7:596-603; EP 244,234; EP 215,594; and Nevalainen et al., “The Molecular Biology of Trichoderma and its Application to the Expression of Both Homologous and Heterologous Genes”, in MOLECULAR INDUSTRIAL MYCOLOGY, Eds. Leong and Berka, Marcel Dekker Inc., NY (1992) pp. 129-148), for transformation of, and heterologous polypeptide expression, in Trichoderma.

In many instances, the introduction of an expression vector into a filamentous fungal cell can involve a process consisting of protoplast formation, transformation of the protoplasts, and regeneration of the strain wall according to methods known in the art. See, e.g., U.S. Pat. No. 7,723,079, Campbell et al., 1989, Curr. Genet. 16:53-56, and Examples below.

In some instances, it is desirable to generate a recombinant filamentous fungal cell in which the expression cassette is integrated in the filamentous fungal genome, as described above. Numerous methods of integrating DNA into filamentous fungal chromosomes are known in the art. Integration of a vector, or portion thereof, into the chromosome of a filamentous fungal cell can be carried out by homologous recombination, non-homologous recombination, or transposition. For applications where site-specific integration is desirable, such as when an expression cassette is generated in the fungal cell genome by operably linking components of an expression cassette to a native gene within the fungal cell's chromosome, vectors typically include targeting sequences that are highly homologous to the sequence flanking the desired site of integration for example as described in Section 4.3. Vectors can include homologous sequence ranging in length from 100 to 1,500 nucleotides, preferably 400 to 1,500 nucleotides, and most preferably 800 to 1,500 nucleotides.

4.6. Use of Recombinant Filamentous Fungal Cells

The recombinant filamentous fungal cells described herein are useful for producing polypeptides of interest. Accordingly, the present disclosure provides methods for producing a polypeptide of interest, comprising culturing a recombinant filamentous fungal cell under conditions that result in expression of the polypeptide of interest. Optionally, the method further comprises additional steps, which can include recovering the polypeptide and purifying the polypeptide.

Suitable filamentous fungal cell culture conditions and culture media are well known in the art. Culture conditions, such as temperature, pH and the like, will be apparent to those skilled in the art. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). Cell culture media in general are set forth in Atlas and Parks (eds.), 1993, The Handbook of Microbiological Media, CRC Press, Boca Raton, Fla., which is incorporated herein by reference. For recombinant expression in filamentous fungal cells, the cells are cultured in a standard medium containing physiological salts and nutrients, such as described in Pourquie et al., 1988, Biochemistry and Genetics of Cellulose Degradation, Aubert et al., eds. Academic Press, pp. 71-86; and Ilmen et al., 1997, Appl. Environ. Microbiol. 63:1298-1306. Culture conditions are also standard, e.g., cultures are incubated at 28° C. in shaker cultures or fermenters until desired levels of polypeptide expression are achieved. Where an inducible promoter is used, the inducing agent, e.g., a sugar, metal salt or antibiotics, is added to the medium at a concentration effective to induce polypeptide expression.

Recombinant filamentous fungal cells may be cultured by shake flask cultivation, small-scale or large-scale fermentation (including continuous, batch, fed-batch, or solid state fermentations) in laboratory or industrial fermentors performed in a suitable medium and under conditions allowing the polypeptide of interest to be expressed and/or isolated.

Techniques for recovering and purifying expressed protein are well known in the art and can be tailored to the particular polypeptide(s) being expressed by the recombinant filamentous fungal cell. Polypeptides can be recovered from the culture medium and or cell lysates. In embodiments where the method is directed to producing a secreted polypeptide, the polypeptide can be recovered from the culture medium. Polypeptides may be recovered or purified from culture media by a variety of procedures known in the art including but not limited to, centrifugation, filtration, extraction, spray-drying, evaporation, or precipitation. The recovered polypeptide may then be further purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation), or extraction (see, e.g., Protein Purification, J.-C. Janson and Lars Ryden, editors, VCH Publishers, New York, 1989).

The recombinant filamentous fungal cells of the disclosure can be used in the production of cellulase compositions. The cellulase compositions of the disclosure typically include a recombinantly expressed POI, which is preferably a cellulase, a hemicellulase or an accessory polypeptide. Cellulase compositions typically include one or more cellobiohydrolases and/or endoglucanases and/or one or more β-glucosidases, and optionally include one or more hemicellulases and/or accessory proteins. In their crudest form, cellulase compositions contain the culture of the recombinant cells that produced the enzyme components. “Cellulase compositions” also refers to a crude fermentation product of the filamentous fungal cells that recombinantly express one or more of a cellulase, hemicellulase and/or accessory protein. A crude fermentation is preferably a fermentation broth that has been separated from the filamentous fungal cells and/or cellular debris (e.g., by centrifugation and/or filtration). In some cases, the enzymes in the broth can be optionally diluted, concentrated, partially purified or purified and/or dried. The recombinant POI produced by the recombinant filamentous fungal cells of the disclosure can be co-expressed with one or more of the other components of the cellulase composition (optionally recombinantly expressed using the same or a different expression cassette of the disclosure) or it can be expressed separately, optionally purified and combined with a composition comprising one or more of the other cellulase components.

Cellulase compositions comprising one or more POIs produced by the recombinant filamentous fungal cells of the disclosure can be used in saccharification reaction to produce simple sugars for fermentation. Accordingly, the present disclosure provides methods for saccharification comprising contacting biomass with a cellulase composition comprising a POI of the disclosure and, optionally, subjecting the resulting sugars to fermentation by a microorganism.

The term “biomass,” as used herein, refers to any composition comprising cellulose (optionally also hemicellulose and/or lignin) As used herein, biomass includes, without limitation, seeds, grains, tubers, plant waste or byproducts of food processing or industrial processing (e.g., stalks), corn (including, e.g., cobs, stover, and the like), grasses (including, e.g., Indian grass, such as Sorghastrum nutans; or, switchgrass, e.g., Panicum species, such as Panicum virgatum), wood (including, e.g., wood chips, processing waste), paper, pulp, and recycled paper (including, e.g., newspaper, printer paper, and the like). Other biomass materials include, without limitation, potatoes, soybean (e.g., rapeseed), barley, rye, oats, wheat, beets, and sugar cane bagasse.

The saccharified biomass (e.g., lignocellulosic material processed by cellulase compositions of the disclosure) can be made into a number of bio-based products, via processes such as, e.g., microbial fermentation and/or chemical synthesis. As used herein, “microbial fermentation” refers to a process of growing and harvesting fermenting microorganisms under suitable conditions. The fermenting microorganism can be any microorganism suitable for use in a desired fermentation process for the production of bio-based products. Suitable fermenting microorganisms include, without limitation, filamentous fungi, yeast, and bacteria. The saccharified biomass can, for example, be made it into a fuel (e.g., a biofuel such as a bioethanol, biobutanol, biomethanol, a biopropanol, a biodiesel, a jet fuel, or the like) via fermentation and/or chemical synthesis. The saccharified biomass can, for example, also be made into a commodity chemical (e.g., ascorbic acid, isoprene, 1,3-propanediol), lipids, amino acids, polypeptides, and enzymes, via fermentation and/or chemical synthesis.

Thus, in certain aspects, POIs expressed by the recombinant filamentous fungal cells of the disclosure find utility in the generation of ethanol from biomass in either separate or simultaneous saccharification and fermentation processes. Separate saccharification and fermentation is a process whereby cellulose present in biomass is saccharified into simple sugars (e.g., glucose) and the simple sugars subsequently fermented by microorganisms (e.g., yeast) into ethanol. Simultaneous saccharification and fermentation is a process whereby cellulose present in biomass is saccharified into simple sugars (e.g., glucose) and, at the same time and in the same reactor, microorganisms (e.g., yeast) ferment the simple sugars into ethanol.

Prior to saccharification, biomass is preferably subject to one or more pretreatment step(s) in order to render cellulose material more accessible or susceptible to enzymes and thus more amenable to hydrolysis by POI polypeptides of the disclosure.

In an exemplary embodiment, the pretreatment entails subjecting biomass material to a catalyst comprising a dilute solution of a strong acid and a metal salt in a reactor. The biomass material can, e.g., be a raw material or a dried material. This pretreatment can lower the activation energy, or the temperature, of cellulose hydrolysis, ultimately allowing higher yields of fermentable sugars. See, e.g., U.S. Pat. Nos. 6,660,506; 6,423,145.

Another exemplary pretreatment method entails hydrolyzing biomass by subjecting the biomass material to a first hydrolysis step in an aqueous medium at a temperature and a pressure chosen to effectuate primarily depolymerization of hemicellulose without achieving significant depolymerization of cellulose into glucose. This step yields a slurry in which the liquid aqueous phase contains dissolved monosaccharides resulting from depolymerization of hemicellulose, and a solid phase containing cellulose and lignin. The slurry is then subject to a second hydrolysis step under conditions that allow a major portion of the cellulose to be depolymerized, yielding a liquid aqueous phase containing dissolved/soluble depolymerization products of cellulose. See, e.g., U.S. Pat. No. 5,536,325.

A further exemplary method involves processing a biomass material by one or more stages of dilute acid hydrolysis using about 0.4% to about 2% of a strong acid; followed by treating the unreacted solid lignocellulosic component of the acid hydrolyzed material with alkaline delignification. See, e.g., U.S. Pat. No. 6,409,841. Another exemplary pretreatment method comprises prehydrolyzing biomass (e.g., lignocellulosic materials) in a prehydrolysis reactor; adding an acidic liquid to the solid lignocellulosic material to make a mixture; heating the mixture to reaction temperature; maintaining reaction temperature for a period of time sufficient to fractionate the lignocellulosic material into a solubilized portion containing at least about 20% of the lignin from the lignocellulosic material, and a solid fraction containing cellulose; separating the solubilized portion from the solid fraction, and removing the solubilized portion while at or near reaction temperature; and recovering the solubilized portion. The cellulose in the solid fraction is rendered more amenable to enzymatic digestion. See, e.g., U.S. Pat. No. 5,705,369. Further pretreatment methods can involve the use of hydrogen peroxide H₂O₂. See Gould, 1984, Biotech, and Bioengr. 26:46-52.

Pretreatment can also comprise contacting a biomass material with stoichiometric amounts of sodium hydroxide and ammonium hydroxide at a very low concentration. See Teixeira et al., 1999, Appl. Biochem. and Biotech. 77-79:19-34. Pretreatment can also comprise contacting a lignocellulose with a chemical (e.g., a base, such as sodium carbonate or potassium hydroxide) at a pH of about 9 to about 14 at moderate temperature, pressure, and pH. See PCT Publication WO2004/081185.

Ammonia pretreatment can also be used. Such a pretreatment method comprises subjecting a biomass material to low ammonia concentration under conditions of high solids. See, e.g., U.S. Patent Publication No. 20070031918 and PCT publication WO 06/110901.

Table 1 below provides a list of the SEQ ID NOs referenced herein and the corresponding polynucleotide or polypeptide sequences.

TABLE 1

| SEQ | | |
|---|---|---|
| ID | | |
| NO: | Description | Sequence |
| 1 | CMV | GTCGTTACAT AACTTACGGT AAATGGCCCG CCTGGCTGAC CGCCCAACGA |
| | promoter | CCCCCGCCCA TTGACGTCAA TAATGACGTA TGTTCCCATA GTAACGCCAA |
| | | TAGGGACTTT CCATTGACGT CAATGGGTGG AGTATTTACG GTAAACTGCC |
| | | CACTTGGCAG TACATCAAGT GTATCATATG CCAAGTACGC CCCCTATTGA |
| | | CGTCAATGAC GGTAAATGGC CCGCCTGGCA TTATGCCCAG TACATGACCT |
| | | TATGGGACTT TCCTACTTGG CAGTACATCT ACGTATTAGT CATCGCTATT |
| | | ACCATGGTGA TGCGGTTTTG GCAGTACATC AATGGGCGTG GATAGCGGTT |
| | | TGACTCACGG GGATTTCCAA GTCTCCACCC CATTGACGTC AATGGGAGTT |
| | | TGTTTTGGCA CCAAAATCAA CGGGACTTTC CAAAATGTCG TAACAACTCC |
| | | GCCCCATTGA CGCAAATGGG CGGTAGGCGT GTACGGTGGG AGGTCTATAT |
| | | AAGCAGAGCT CGTTTAGTGA ACCGT |
| 2 | T. reesei | CCTCCTCCCT CTCTCCCTCT CGTTTCTTCC TAACAAACAA CCACCACCAA |
| | gpd 5′ UTR | AATCTCTTTG GAAGCTCACG ACTCACGCAA GCTCAATTCG CAGATACAAA |
| | 100 nt | |
| | fragment | |
| 3 | T. reesei | AGCTACCCCG CCAGACTCTC CTGCGTCACC AATTTTTTTC CCTATTTACC |
| | gpd 5′ UTR | CCTCCTCCCT CTCTCCCTCT CGTTTCTTCC TAACAAACAA CCACCACCAA |
| | 150 nt | AATCTCTTTG GAAGCTCACG ACTCACGCAA GCTCAATTCG CAGATACAAA |
| | fragment | |
| 4 | T. reesei | ACGATGCGGC TTCTGTTCGC CTGCCCCTCC TCCCACTCGT GCCCTTGACG |
| | gpd 5′ UTR | AGCTACCCCG CCAGACTCTC CTGCGTCACC AATTTTTTTC CCTATTTACC |
| | 200 nt | CCTCCTCCCT CTCTCCCTCT CGTTTCTTCC TAACAAACAA CCACCACCAA |
| | fragment | AATCTCTTTG GAAGCTCACG ACTCACGCAA GCTCAATTCG CAGATACAAA |
| 5 | T. reesei | GGCCAGGTCC TGAACCCTTA CTACTCTCAG TGCCTGTAAA GCTCCGTGGC |
| | CBHI | GAAAGCCTGA CGCACCGGTA GATTCTTGGT GAGCCCGTAT CATGACGGCG |
| | terminator | GCGGGAGCTA CATGGCCCCG GGTGATTTAT TTTTTTTGTA TCTACTTCTG |
| | | ACCCTTTTCA AATATACGGT CAACTCATCT TTCACTGGAG ATGCGGCCTG |
| | | CTTGGTATTG CGATGTTGTC AGCTTGGCAA ATTGTGGCTT TCGAAAACAC |
| | | AAAACGATTC CTTAGTAGCC ATGCATTTTA AGATAACGGA ATAGAAGAAA |
| | | GAGGAAATTA AAAAAAAAAA AAAACAAACA TCCCGTTCAT AACCCGTAGA |
| | | ATCGCCGCTC TTCGTGTATC CCAGTACCAC GGCAAAGGTA TTTCATGATC |
| | | GTTCAATGTT GATATTGTTC CCGCCAGTAT GGCTCCACCC CCATCTCCGC |
| | | GAATCTCCTC TTCTCGAACG CGGTAGTGGC GCGCCAATTG GTAATGACCC |
| | | ATAGGGAGAC AAACAGCATA ATAGCAACAG TGGAAATTAG TGGCGCAATA |
| | | ATTGAGAACA CAGTGAGACC ATAGCTGGCG GCCTGGAAAG CACTGTTGGA |
| | | GACCAACTTG TCCGTTGCGA GGCCAACTTG CATTGCTGTC AGGACGATGA |
| | | CAACGTAGCC GAGGACCGTC ACAAGGGACG CAAGTGCG |
| 6 | CMV | CACCATTAAT TAAGTCGTTA CATAACTTAC GGTAAATGGC CC |
| | promoter | |
| | forward | |
| | primer | |
| 7 | CMV | CACCACGGAC CGTACTAGTA CGGTTCACTA AACGAGCTCT GC |
| | promoter | |
| | reverse | |
| | primer | |
| 8 | β-glucosidase | CACCAACTAG TATGCTGTGG CTTGCACAAG CATTGTTGG |
| | forward | |
| | primer | |
| 9 | β-glucosidase | CACCAGGCCG GCCTTATCTA AAGCTGCTAG TGTCCAGTG GGG |
| | reverse | |
| | primer | |
| 10 | TR-CBHIt- | ACTTTGCGTC CCTTGTGACG G |
| | 3′ primer | |
| 11 | TR-PYR4- | TTGCATTGGT ACAGCTGCAG G |
| | 5′ primer | |
| 12 | 5′ UTR | GACTCACGCA AGCTCAATTC G |
| | forward | |
| | primer, | |
| | −34 from | |
| | ATG start | |
| 13 | 5′ UTR | CCAGACTCTC CTGCGTCACC AAT |
| | forward | |
| | primer, | |
| | −140 from | |
| | ATG start | |
| 14 | 5′ UTR | CTACAATCAT CACCACGATG CTCC |
| | forward | |
| | primer, | |
| | −229 from | |
| | ATG start | |
| 15 | 5′ UTR | CGACATTCTC TCCTAATCAC CAGC |
| | forward | |
| | primer, | |
| | −284 from | |
| | ATG start | |
| 16 | 5′ UTR | GCCGTGCCTA CCTGCTTTAG TATT |
| | forward | |
| | primer, | |
| | −402 from | |
| | ATG start | |
| 17 | 5′ UTR | CCACTATCTC AGGTAACCAG GTAC |
| | forward | |
| | primer, | |
| | −443 from | |
| | ATG start | |
| 18 | Reverse | GTCTCGCTCC ACTTGATGTT GGCA |
| | primer, | |
| | +269 from | |
| | ATG start | |
| 19 | CMV | CAGATCGCCT GGAGACGCCA TCCACGCTGT TTTGACCTCC ATAGAAGACA |
| | native | CCGGGACCGA TCCAGCCTCC GCGGCCGGGA ACGGTGCATT GGAACGCGGA |
| | 5′ UTR | TTCCCCGTGC CAAGAGTGAC GTAAGTACCG CCTATAGAGT CTATAGGCCC |
| | | ACCCCCTTGG CTTCTTATGC |
| 20 | pC forward | CACCATTAAT TAAGTCGTTA CATAACTTAC GGTAAATGG |
| | primer | |
| | with PacI | |
| | site | |
| 21 | pCMV3′ + | AGGTCAAAAC AGCGTGGATG GCGTCTCCAG GCGATCTGAC GGTTCACTAAA |
| | UTR1 | CGAGCTCTG |
| 22 | pC-5′UTR- | CGGCCGCGGAG GCTGGATCG GTCCCGGTGT CTTCTATGGA GGTCAAAACA |
| | Reverse 1 | GCGTGGATGG |
| 23 | pC-5′UTR- | ACTCTTGGCA CGGGGAATCC GCGTTCCAAT GCACCGTTCC CGGCCGCGGA |
| | Reverse 2 | GGCTGGATCG |
| 24 | pC-5′UTR- | AGGGGGTGGG CCTATAGACT CTATAGGCGG TACTTACGTC ACTCTTGGCA |
| | Reverse 3 | CGGGGAATCC |
| 25 | pC-5′UTR- | CACCAACTAG TGCATAAGAA GCCAAGGGGG TGGGCCTATA GACTC |
| | Reverse 4 | |
| 26 | pC overlap- | GCGAACAGAA GCCGCATCGT ACGGTTCACT AAACGAGCTC |
| | 100 bp gpd | |
| | 5′ UTR | |
| | reverse | |
| | primer | |
| 27 | pC overlap- | GAGAGTCTGG CGGGGTAGCT ACGGTTCACT AAACGAGCTC |
| | 150 bp gpd | |
| | 5′ UTR | |
| | reverse | |
| | primer | |
| 28 | pC overlap- | GCGAACAGAA GCCGCATCGT ACGGTTCACT AAACGAGCTC |
| | 200 bp gpd | |
| | 5′ UTR | |
| | reverse | |
| | primer | |
| 29 | pC overlap- | GAGCTCGTTT AGTGAACCGT ACGATGCGGC TTCTGTTCGC |
| | 100 bp gpd | |
| | 5′ UTR | |
| | forward | |
| | primer | |
| 30 | pC overlap- | GAGCTCGTTT AGTGAACCGT AGCTACCCCG CCAGACTCTC |
| | 150 bp gpd | |
| | 5′ UTR | |
| | forward | |
| | primer | |
| 31 | pC overlap- | GAGCTCGTTT AGTGAACCGT ACGATGCGGCT TCTGTTCGC |
| | 200 bp gpd | |
| | 5′ UTR | |
| | forward | |
| | primer | |
| 32 | pWG-SpeI | CACCAACTA GTTTTGTATCT GCGAATTGAG CTTGCGTGA |
| | site | |
| | reverse | |
| | primer | |
| 33 | Cochliobolus | ATGCTGTGGC TTGCACAAGC ATTGTTGGTC GGCCTTGCCC AGGCATCGCC |
| | heterostrophus | CAGGTTCCCT CGTGCTACCA ACGACACCGG CAGTGATTCT TTGAACAATG |
| | β-glucosidase | CCCAGAGCCC GCCATTCTAC CCAAGTCCTT GGGTAGATCC CACCACCAAG |
| | nucleotide | GACTGGGCGG CTGCCTATGA AAAAGCAAAG GCTTTTGTTA GCCAATTGAC |
| | sequence | TCTTATTGAG AAGGTCAACC TCACCACCGG CACTGGATGG CAGAGCGACC |
| | | ACTGCGTTGG TAACGTGGGC GCTATTCCTC GCCTTGGCTT TGATCCCCTC |
| | | TGCCTCCAGG ACAGCCCTCT CGGCATCCGT TTCGCAGACT ACGTTTCTGC |
| | | TTTCCCAGCA GGTGGCACCA TTGCTGCATC ATGGGACCGC TATGAGTTTT |
| | | ACACCCGCGG TAACGAGATG GGTAAGGAGC ACCGAAGGAA GGGAGTCGAC |
| | | GTTCAGCTTG GTCCTGCCAT TGGACCTCTT GGTCGCCACC CCAAGGGCGG |
| | | TCGTAACTGG GAAGGCTTCA GTCCTGATCC TGTACTTTCC GGTGTGGCCG |
| | | TGAGCGAAAC AGTCCGCGGT ATCCAGGATG CTGGTGTCAT TGCCTGCACT |
| | | AAGCACTTCC TTCTGAACGA GCAAGAACAT TTCCGTCAGC CCGGCAGTTT |
| | | CGGAGATATC CCCTTTGTCG ATGCCATCAG CTCCAATACC GATGACACGA |
| | | CTCTACACGA GCTCTACCTG TGGCCCTTTG CCGACGCCGT CCGCGCTGGT |
| | | ACTGGTGCCA TCATGTGCTC TTACAACAAG GCCAACAACT CGCAACTCTG |
| | | CCAAAACTCG CACCTTCAAA ACTATATTCT CAAGGGCGAG CTTGGCTTCC |
| | | AGGGTTTCAT TGTATCTGAC TGGGATGCAC AGCACTCGGG CGTTGCGTCG |
| | | GCTTATGCTG GATTGGACAT GACTATGCCT GGTGATACTG GATTCAACAC |
| | | TGGACTGTCC TTCTGGGGCG CTAACATGAC CGTCTCCATT CTCAACGGCA |
| | | CCATTCCCCA GTGGCGTCTC GACGATGCGG CCATCCGTAT CATGACCGCA |
| | | TACTACTTTG TCGGCCTTGA TGAGTCTATC CCTGTCAACT TTGACAGCTG |
| | | GCAAACTAGC ACGTACGGAT TCGAGCATTT TTTCGGAAAG AAGGGCTTCG |
| | | GTCTGATCAA CAAGCACATT GACGTTCGCG AGGAGCACTT CCGCTCCATC |
| | | CGCCGCTCTG CTGCCAAGTC AACCGTTCTC CTCAAGAACT CTGGCGTCCT |
| | | TCCCCTCTCT GGAAAGGAGA AGTGGACTGC TGTATTTGGA GAAGATGCTG |
| | | GCGAAAACCC GCTGGGCCCC AACGGATGCG CTGACCGCGG CTGCGACTCT |
| | | GGCACCTTGG CCATGGGCTG GGGTTCGGGA ACTGCAGACT TCCCTTACCT |
| | | CGTCACTCCT CTCGAAGCCA TCAAGCGTGA GGTTGGCGAG AATGGCGGCG |
| | | TGATCACTTC GGTCACAGAC AACTACGCCA CTTCGCAGAT CCAGACCATG |
| | | GCCAGCAGGG CCAGCCACTC GATTGTCTTC GTCAATGCCG ACTCTGGTGA |
| | | AGGTTACATC ACTGTTGATA ACAACATGGG TGACCGCAAC AACATGACTG |
| | | TGTGGGGCAA TGGTGATGTG CTTGTCAAGA ATATCTCTGC TCTGTGCAAC |
| | | AACACGATTG TGGTTATCCA CTCTGTCGGC CCAGTCATTA TTGACGCCTG |
| | | GAAGGCCAAC GACAACGTGA CTGCCATTCT CTGGGCTGGT CTTCCTGGCC |
| | | AGGAGTCTGG TAACTCGATT GCTGACATTC TATACGGACA CCACAACCCT |
| | | GGTGGCAAGC TCCCCTTCAC CATTGGCAGC TCTTCAGAGG AGTATGGCCC |
| | | TGATGTCATC TACGAGCCCA CGAACGGCAT CCTCAGCCCT CAGGCCAACT |
| | | TTGAAGAGGG CGTCTTCATT GACTACCGCG CGTTTGACAA GGCGGGCATT |
| | | GAGCCCACGT ACGAATTTGG CTTTGGTCTT TCGTACACGA CTTTTGAATA |
| | | CTCGGACCTC AAGGTCACTG CGCAGTCTGC CGAGGCTTAC AAGCCTTTCA |
| | | CCGGCCAGAC TTCGGCTGCC CCTACATTCG GAAACTTCAG CAAGAACCCC |
| | | GAGGACTACC AGTACCCTCC CGGCCTTGTT TACCCCGACA CGTTCATCTA |
| | | CCCCTACCTC AACTCGACTG ACCTCAAGAC GGCATCTCAG GATCCCGAGT |
| | | ACGGCCTCAA CGTTACCTGG CCCAAGGGCT CTACCGATGG CTCGCCTCAG |
| | | ACCCGCATTG CGGCTGGTGG TGCGCCCGGC GGTAACCCCC AGCTCTGGGA |
| | | CGTTTTGTTC AAGGTCGAGG CCACGATCAC CAACACTGGT CACGTTGCTG |
| | | GTGACGAGGT GGCCCAGGCG TACATCTCGC TTGGTGGCCC CAACGACCCC |
| | | AAGGTGCTAC TCCGTGACTT TGACCGCTTG ACCATCAAGC CTGGTGAGAG |
| | | CGCTGTTTTC ACAGCCAACA TCACCCGCCG TGATGTCAGC AACTGGGACA |
| | | CTGTCAGCCA GAACTGGGTC ATTACCGAGT ACCCCAAGAC GATCCACGTT |
| | | GGTGCCAGTT CGAGGAACCT TCCTCTTTCT GCCCCACTGG ACACTAGCAG |
| | | CTTTAGATAA |
| 34 | Cochliobolus | MLWLAQALLV GLAQASPRFP RATNDTGSDS LNNAQSPPFY PSPWVDPTTK |
| | heterostrophus | DWAAAYEKAK AFVSQLTLIE KVNLTTGTGW QSDHCVGNVG AIPRLGFDPL |
| | β-glucosidase | CLQDSPLGIR FADYVSAFPA GGTIAASWDR YEFYTRGNEM GKEHRRKGVD |
| | polypeptide | VQLGPAIGPL GRHPKGGRNW EGFSPDPVLS GVAVSETVRG IQDAGVIACT |
| | sequence | KHFLLNEQEH FRQPGSFGDI PFVDAISSNT DDTTLHELYL WPFADAVRAG |
| | | TGAIMCSYNK ANNSQLCQNS HLQNYILKGE LGFQGFIVSD WDAQHSGVAS |
| | | AYAGLDMTMP GDTGFNTGLS FWGANMTVSI LNGTIPQWRL DDAAIRIMTA |
| | | YYFVGLDESI PVNFDSWQTS TYGFEHFFGK KGFGLINKHI DVREEHFRSI |
| | | RRSAAKSTVL LKNSGVLPLS GKEKWTAVFG EDAGENPLGP NGCADRGCDS |
| | | GTLAMGWGSG TADFPYLVTP LEAIKREVGE NGGVITSVTD NYATSQIQTM |
| | | ASRASHSIVF VNADSGEGYI TVDNNMGDRN NMTVWGNGDV LVKNISALCN |
| | | NTIVVIHSVG PVIIDAWKAN DNVTAILWAG LPGQESGNSI ADILYGHHNP |
| | | GGKLPFTIGS SSEEYGPDVI YEPTNGILSP QANFEEGVFI DYRAFDKAGI |
| | | EPTYEFGFGL SYTTFEYSDL KVTAQSAEAY KPFTGQTSAA PTFGNFSKNP |
| | | EDYQYPPGLV YPDTFIYPYL NSTDLKTASQ DPEYGLNVTW PKGSTDGSPQ |
| | | TRIAAGGAPG GNPQLWDVLF KVEATITNTG HVAGDEVAQA YISLGGPNDP |
| | | KVLLRDFDRL TIKPGESAVF TANITRRDVS NWDTVSQNWV ITEYPKTIHV |
| | | GASSRNLPLS APLDTSSFR |

### 5. EXAMPLES

### 5.1. Example 1

### Construction of a Vector Containing a CMV Promoter Sequence and the Coding Sequence for Cochliobolus Heterostrophus β-Glucosidase

This example describes the construction of an expression vector comprising a cytomegalovirus (CMV) promoter operably linked in a 5′ to 3′ direction to a sequence coding for Cochliobolus heterostrophus β-glucosidase and a terminator sequence from T reesei CBHI, which includes a 3′ UTR.

Construction of Plasmids Containing CMV Promoter.

First, vectors containing a cytomegalovirus (CMV) promoter were constructed by inserting the viral CMV promoter into plasmid pW, which consists of the commercial plasmid pBluescript II SK (+), the Trichoderma reesei selectible marker PYR4 (encoding orotidine-5′-monophosphate decarboxylase) and the terminator from CBHI (encoding exo-cellobiohydrolase I). All procedures utilizing commercial vendor products, described in this and the following Examples, were carried out by following the instructions of the manufacturer. The vector containing CMV promoter is denominated pC. The promoter was cloned into the plasmid using conventional techniques. The promoter was amplified by polymerase chain reaction (PCR) from a synthesized template with AccuPrime™ Pfx SuperMix (Invitrogen, Carlsbad, Calif.) using the primers listed below.

TABLE 2

| SEQ | | |
|---|---|---|
| ID | | |
| NO: | Description | Sequence |
| 6 | CMV forward (5′) | CACCATTAATTAAGTCGTTACATAACTTACGGTAAATGGCCC |
| | primer | |
| 7 | CMV reverse (3′) | CACCACGGACCGTACTAGTACGGTTCACTAAACGAGCTCTGC |
| | primer | |

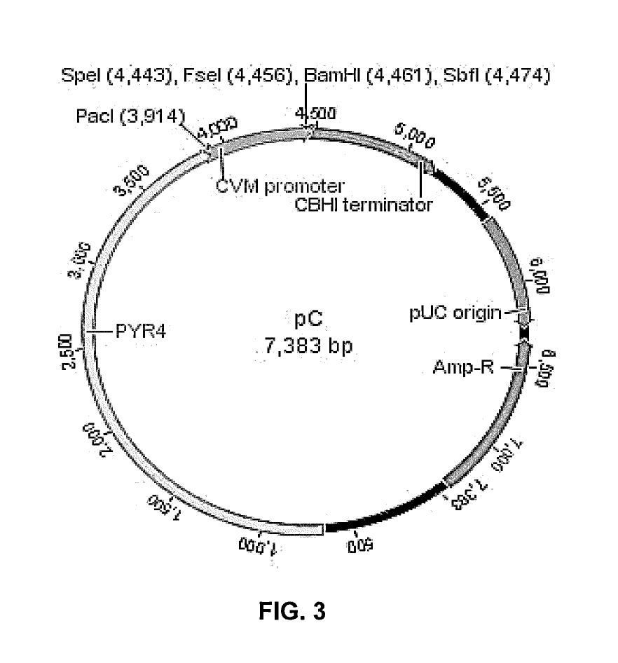

Each primer contains a CACCA sequence of nucleotides on its 5′ end to ensure efficient cutting. The forward primer contains a PacI restriction site and the reverse primer contains an RsrII restriction site as well as a SpeI restriction site. In the table above, restriction sites are underlined. The amplified promoter was then purified with the DNA Clean & Concentrator™5 kit (Zymo Research, Irvine, Calif.), digested with PacI and SpeI (NEB, Ipswich, Mass.); gel purified with Zymoclean™ Gel DNA Recovery Kit (Zymo Research, Irvine, Calif.) to prepare the promoter DNA for ligation. Plasmid DNA was prepared by digesting pW with PacI and SpeI at 37° C. for 2 hours and then purified with the DNA Clean & Concentrator™-5 kit. The ligation reaction between the promoter DNA and the plasmid DNA was carried with T4 DNA Ligase (NEB, Ipswich, Mass.). Each 10 μL ligation consisted of 50 ng of plasmid DNA, 20 ng or 40 ng of promoter DNA (so that promoter to vector molar ratio is 5:1), 1× T4 DNA Ligase buffer and 0.2 μL T4 DNA ligase. The sequence of the inserted promoter was verified by sequencing using Big-Dye™ terminator chemistry (Applied Biosystems, Inc., Foster City, Calif.). FIG. 3A depicts a schematic map of the resulting pC vector.

Construction of Vector Containing a Cochliobolus Heterostrophus β-Glucosidase Coding Sequence.

The pC vector was digested with SpeI and FseI at 37° C. for 2 hours and purified with the DNA Clean & Concentrator™5 kit. Sequences encoding a β-glucosidase were amplified using AccuPrime™ Pfx SuperMix with the primers listed below.

TABLE 3

| SEQ | | |
|---|---|---|
| ID | | |
| NO: | Description | Sequence |
| 8 | β-glucosidase | CACCAACTAGTATGCTGTGGCTTGCACAAGCATTGTTGG |
| | forward (5′) | |
| | primer | |
| 9 | β-glucosidase | CACCAGGCCGGCCTTATCTAAAGCTGCTAGTGTCCAGTGGGG |
| | reverse (3′) | |
| | primer | |

Primers were designed to have a melting temperature (T_{M}) of 60° C., a CACCA sequence on their 5′ end to ensure efficient cutting in subsequent steps. The forward primer then included a SpeI restriction site and the reverse primer an FseI restriction site to allow for cloning into the pC vector. Restriction sites are underlined and the sequence corresponding to the β-glucosidase coding sequence is shown in italics in the table above. The amplified coding sequence was then purified with the DNA Clean & Concentrator™-5 (Zymo Research, Irvine, Calif.) digested with PacI and SpeI (NEB, Ipswich, Mass.); gel purified with Zymoclean™ Gel DNA Recovery Kit (Zymo Research, Irvine, Calif.) to prepare the coding sequence DNA for ligation. Ligation was carried out using T4 DNA Ligase (NEB, Ipswich, Mass.). Each 10 μL ligation consisted of 50 ng of pC vector, 20 ng or 40 ng of coding sequence DNA (so that coding sequence to pC vector molar ratio is 5:1), 1× T4 DNA Ligase buffer and 0.2 μL T4 DNA Ligase. The nucleotide sequences of the final constructs were confirmed using Big-Dye™ terminator chemistry (Applied Biosystems, Inc., Foster City, Calif.). The plasmid containing the CMV promoter operably linked to β-glucosidase is denominated pC-BG.

### 5.2. Example 2

### Transformation of Trichoderma Reesei with Vector Containing a CMV Promoter and a Protein Coding Sequence

This example describes the introduction of an expression vector comprising a CMV promoter operably linked in a 5′ to 3′ direction to a protein coding sequence for Cochliobolus heterostrophus β-glucosidase.

Media.

The following media was used for the transformation procedure. Aspergillus Complete Medium (ACM) was made as follows: 10 g/l yeast extract (1% final); 25 g/l glucose (2.5% final); 10 g/l Bacto Peptone (Bacto Laboratories, Liverpool, NSW, Australia) (1% final); 7 mM KCl; 11 mM KH₂PO₄; 2 mM MgSO₄; 77 μM ZnSO₄; 178 μM H₃BO₃; 25 μM MnCl₂; 18 μM FeSO₄; 7.1 μM CoCl₂; 6.4 μM CuSO₄; 6.2 μM Na₂MoO₄; 134 μM Na₂EDTA; 1 mg/ml riboflavin; 1 mg/ml thiamine; 1 mg/ml nicotinamide; 0.5 mg/ml pyridoxine; 0.1 mg/ml pantothenic acid; 2 μg/ml biotin. Trichoderma Minimal Medium (TMM) plates were made as follows: 10 g/l glucose; 45 mM (NH₄)₂SO₄; 73 mM KH₂PO₄; 4 mM MgSO₄; 10 mM trisodium citrate; 18 μM FeSO₄; 10 μM MnSO₄; 5 μM ZnSO₄; 14 μM CaCl₂; 15 g/l agar (TMM overlay contains 7.5 g/l agar).

Amplification of pC-BG DNA.

The amplification reactions (50 μl) were set up to contain 1× AccuPrime Pfx Supermix (Invitrogen, Carlsbad, Calif.), 0.28 μM primer TR-CBHIt-3′ (ACTTTGCGTCCCTTGTGACGG) (SEQ ID NO:10), 0.28 μM primer TR-PYR4-5′ (TTGCATTGGTACAGCTGCAGG) (SEQ ID NO:11), and 30-40 ng of pC-BG DNA. The reactions were subjected to thermocyling in a GeneAmp 9700 (Applied Biosystems, Carlsbad, Calif.) programmed as follows: 95° C. for 3 minutes, then 30 cycles each of 45 seconds at 95° C., 45 seconds at 57° C., and 8.5 minutes at 68° C. (with a 10 minute final extension at 68° C.). The reaction products were visualized on a ReadyAgrose gel (Bio-Rad, Hercules, Calif.) and purified using a QIAquick PCR purification kit (Qiagen, Valencia, Calif.) according to the manufacturer's instructions.

Transformation of Trichoderma Reesei.

A pyr4-deficient mutant of Trichoderma reesei strain MCG80 was used as the expression host for the pC-BG construct, allowing for pyr4 selection of transformants Mycelial cultures of MCG80pyr4 were produced by adding 2.2×10⁸ conidia to 400 ml ACM medium and incubating in an orbital shaking incubator at 30° C. and 275 rpm for 18 hrs. Mycelia were gently washed with 450 ml of KM (0.7 M KCl; 20 mM MES buffer, pH 6.0) using a sterile 1-liter filter unit. Washed mycelia were suspended in 100 ml of KM containing 15 mg/ml Lysing Enzymes from Trichoderma harzianum (Sigm-Aldrich, St. Louis, Mo.) and incubated in an orbital shaker at 30° C. and 60 rpm for 90 minutes. Mycelial debris was removed from the protoplast suspension by filtering through Miracloth (EMD Biosciences, Gibbstown, N.J.). The resulting suspension was transferred to a 250 ml centrifuge bottle and filled to the top with ice cold STC (1 M sorbitol; 50 mM CaCl₂; 10 mM Tris-HCl, pH 7.5), mixed and centrifuged (15 min, 2100×g, 4° C.). After discarding the supernatant, the pellet was gently suspended in 250 ml ice cold STC and centrifuged again (15 min, 2100×g, 4° C.). The resulting pellet was suspended in STC at a concentration of approximately 5×10⁷ protoplasts per ml, based on hemacytometer count.

For each filamentous fungal transformation, a 200 μl aliquot of protoplast suspension was added to a 15 ml test tube and incubated at 50° C. for 1 min then rapidly cooled on ice. Following a 5 min incubation at room temperature, 20 μl of PCR-amplified pC-BG DNA (containing the mammalian viral promoter, β-glucosidase coding sequence and the pyr4 selectable marker) was added, along with 20 μl 0.2 M ammonium aurintricarboxylate (Sigma-Aldrich, St. Louis, Mo.) and 50 μl PEG buffer (60% polyethylene glycol 4000; 50 mM CaCl₂; 10 mM Tris-HCl, pH 7.5) and mixed well. The tube was heat-shocked again at 50° C. for 1 min, quickly cooled on ice, then incubated at room temperature for 20 min. Another 1.5 ml of PEG buffer was then added and mixed thoroughly by carefully rotating the tube. After a final 5 min incubation at room temperature, 5 ml of ice cold STC was added to the tube and mixed by inversion. The sample was then centrifuged (10 min, 3300×g, 4° C.) and the resulting pellet was suspended in approximately 500 μl of ice cold STC. A soft agar overlay technique was used to plate the transformation suspension onto selective media (TMM) osmotically stabilized with 0.6 M KCl. Plates were incubated at 30° C. Colonies of transformants were typically visible after 5-6 days.

### 5.3. Example 3

### Identification of 5′ UTR for T. reesei Glyceraldehyde-3-Phosphate Dehydrogenase (Gpd) Gene

This example describes the mapping of 5′ untranslated sequence in the Trichoderma reesei gpd gene.

In order to determine the approximate 5′UTR transcript initiation point, nested forward primers were designed within the 5′ upstream region of the gpd gene. Standard PCR with each of these primers paired with a gpd coding sequence reverse primer was conducted on both cDNA (variable) and gDNA (control) sample templates for the Trichoderma reesei strain MCG80. Reverse-Transcriptase PCR (RT-PCR) was used to amplify the 5′ UTR from the gpd gene from Trichoderma reesei RNA. Total RNA was extracted from Trichoderma reesei MCG80 culture using RNeasy Plant Mini Kit (Qiagen, Valencia, Calif.) and was used as template for RT-PCR/cDNA synthesis using Verso cDNA synthesis kit (Thermo Fisher Scientific, Fremont, Calif.) and subsequent PCR reactions. Genomic DNA (gDNA) was extracted from MCG80 culture using Masterpure Yeast DNA Purification Kit (Epicentre, Madison, Wisc.) and was used as template for control PCR reactions.

The following primers were used.

TABLE 4

| SEQ | | | |
|---|---|---|---|
| ID | Primer | | |
| NO: | # | Description | Sequence |
| 12 | 1 | 5′ UTR | GACTCACGCAAGCTCAATTCG |
| | | forward | |
| | | primer, −34 | |
| | | from ATG | |
| | | start | |
| 13 | 2 | 5′ UTR | CCAGACTCTCCTGCGTCACCAAT |
| | | forward | |
| | | primer, −140 | |
| | | from ATG | |
| | | start | |
| 14 | 3 | 5′ UTR | CTACAATCATCACCACGATGCTCC |
| | | forward | |
| | | primer, −229 | |
| | | from ATG | |
| | | start | |
| 15 | 4 | 5′ UTR | CGACATTCTCTCCTAATCACCAGC |
| | | forward | |
| | | primer, −284 | |
| | | from ATG | |
| | | start | |
| 16 | 5 | 5′ UTR | GCCGTGCCTACCTGCTTTAGTATT |
| | | forward | |
| | | primer, −402 | |
| | | from ATG | |
| | | start | |
| 17 | 6 | 5′ UTR | CCACTATCTCAGGTAACCAGGTAC |
| | | forward | |
| | | primer, −443 | |
| | | from ATG | |
| | | start | |
| 18 | 7 | Reverse | GTCTCGCTCCACTTGATGTTGGCA |
| | | primer, +269 | |
| | | from ATG | |
| | | start | |

The following forward and reverse primer combinations were run with both cDNA and gDNA templates.

Reaction #1 cDNA template with primer 1+primer 7

Reaction #2 cDNA template with primer 2+primer 7

Reaction #3 cDNA template with primer 3+primer 7

Reaction #4 cDNA template with primer 4+primer 7

Reaction #5 cDNA template with primer 5+primer 7

Reaction #6 cDNA template with primer 6+primer 7

Reaction #7 gDNA template with primer 1+primer 7

Reaction #8 gDNA template with primer 2+primer 7

Reaction #9 gDNA template with primer 3+primer 7

Reaction #10 gDNA template with primer 4+primer 7

Reaction #11 gDNA template with primer 5+primer 7

Reaction #12 gDNA template with primer 6+primer 7

The PCR reactions were prepared in 25 μl volumes containing the following: 9.5 μl water, 12.5 μl Taq polymerase mix, 1 μl each of the specified forward and reverse primer (1 μM), and 1 μl of the appropriate template DNA. The following thermal cycling steps were carried out: a cycle at 95° C. for 5 minutes, followed by 30 cycles of three steps consisting of 95° C. for 30 seconds, followed by 55° C. for 30 second, followed by 72° C. for 1 minutes, and ending with a 7 minute cycle at 72° C. 10 μl of each reactions were run on a 1% agarose gel. Bands were excised and purified using a Zymo Research Gel Extraction Kit (Zymo Research, Irvine, Calif.). The resulting fragments were cloned into pCR4-TOPO using a TOPO cloning for sequencing kit (Invitrogen, Carlsbad, Calif.) following the manufacturer's protocol. Individual clones were submitting for full length insert sequencing.

Results.

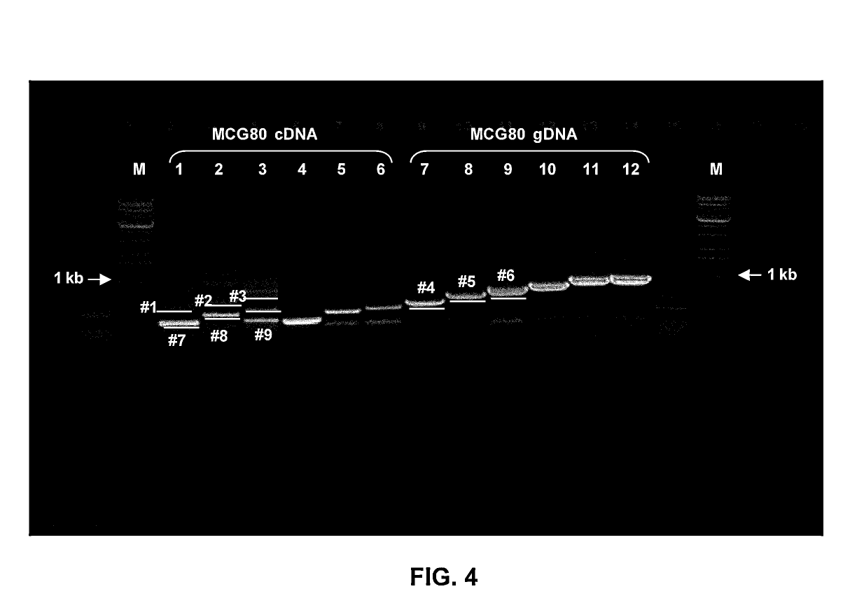

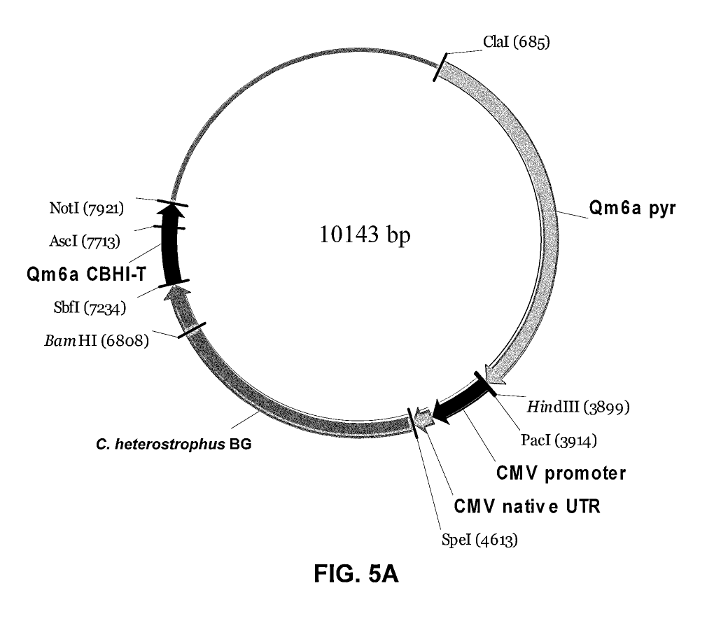

As shown in FIG. 4, cDNA reaction banding patterns were compared to the counterpart reaction for the gDNA control. In this way, banding patterns would indicate that the standard PCR reaction for the nested set falls off between −229 and −284 bp upstream of the ATG start site. The genomic reaction banding pattern forms a steady nested pattern progression which is not seen for the cDNA sample set. Due to possible intron sites present in the gDNA template, the first three lanes for cDNA and corresponding gDNA reactions may not match exactly in size. Based on the observed banding patterns and sequence data results, indications are that the 5′UTR initiation site for the Trichoderma reesei MCG80 strain gpd transcript is between −229 and −284 bp upstream of the ATG start site. The appropriate bands, based on the upward nested banding pattern alone, were selected for excision. The sequence of the 5′ UTR gpd fragments used to construct expression cassettes is as follows.

TABLE 5

| SEQ | | |
|---|---|---|
| ID | | |
| NO: | Description | Sequence |
| 2 | 100 bp gpd | CCTCCTCCCT CTCTCCCTCT CGTTTCTTCC TAACAAACAA |
| | 5′UTR | CCACCACCAA AATCTCTTTG GAAGCTCACG ACTCACGCAA |
| | | GCTCAATTCG CAGATACAAA |
| 3 | 150 bp gpd | AGCTACCCCG CCAGACTCTC CTGCGTCACC AATTTTTTTC |
| | 5′UTR | CCTATTTACC CCTCCTCCCT CTCTCCCTCT CGTTTCTTCC |
| | | TAACAAACAA CCACCACCAA AATCTCTTTG GAAGCTCACG |
| | | ACTCACGCAA GCTCAATTCG CAGATACAAA |
| 4 | 200 bp gpd | ACGATGCGGC TTCTGTTCGC CTGCCCCTCC TCCCACTCGT |
| | 5′UTR | GCCCTTGACG AGCTACCCCG CCAGACTCTC CTGCGTCACC |
| | | AATTTTTTTC CCTATTTACC CCTCCTCCCT CTCTCCCTCT |
| | | CGTTTCTTCC TAACAAACAA CCACCACCAA AATCTCTTTG |
| | | GAAGCTCACG ACTCACGCAA GCTCAATTCG CAGATACAAA |

### 5.4. Example 4

### Construction of a Vector Containing an Expression Cassette Including a CMV Promoter, a 5′ Untranslated Region (5′ UTR), and the Protein Coding Sequence for Cochliobolus Heterostrophus β-Glucosidase

This example describes the construction of expression cassettes comprising a CMV promoter, a 5′ UTR from CMV or from the Trichoderma reesei gpd gene, and the protein coding sequence for Cochliobolus heterostrophus β-glucosidase, and a CBHI terminator as the 3′ UTR.

The DNA fragments of CMV promoter linked to a 5′UTR were generated using an ‘overlapping PCR’ strategy and then cloned into the pC vector. 5′ UTR sequence from gpd was amplified from pWG, a plasmid derived from pW described above incorporating the native gpd promoter from Trichoderma reesei. The plasmid pC provided the template DNA for the CMV promoter.

5′ UTR sequences used to generate expression cassettes is as follows for native CMV 5′UTR: CAGATCGCCT GGAGACGCCA TCCACGCTGT TTTGACCTCC ATAGAAGACA CCGGGACCGA TCCAGCCTCCG CGGCCGGGAA CGGTGCATTGG AACGCGGATTC CCCGTGCCAAG AGTGACGTAAG TACCGCCTATA GAGTCTATAGG CCCACCCCCTT GGCTTCTTATGC (SEQ ID NO:19), and as provided in Table 4 in Example 3 above for each of the 5′ UTR from gpd.

Construction of CMV Promoter with Native CMV 5′ UTR.

The CMV promoter fragment was also extended to incorporate sequences from the UTR of the native CMV transcript. The PCR template DNA for the amplification of the CMV promoter was plasmid pC as described above. The PCR primers used to construct a sequence including the CMV promoter and the native CMV 5′UTR were as follows:

TABLE 6

| SEQ | | |
|---|---|---|
| ID | | |
| NO: | Description | Sequence |
| 20 | pC forward primer | CACCATTAATTAAGTCGTTACATAACTTACGGTAAATGG |
| | with PacI site | |
| 21 | pCMV3′ end of | AGGTCAAAAC AGCGTGGATG GCGTCTCCAG |
| | 5′UTR reverse | GCGATCTGAC GGTTCACTAAA CGAGCTCTG |
| | primer | |
| 22 | pC-5′UTR- | CGGCCGCGGAG GCTGGATCGGT CCCGGTGTCTTC |
| | Reverse 1 | TATGGAGGTCA AAACAGCGTGG ATGG |
| | primer | |
| 23 | pC-5′UTR- | ACTCTTGGCACG GGGAATCCGCG TTCCAATGCACC |
| | Reverse 2 | GTTCCCGGCCGC GGAGGCTGGAT CG |
| | primer | |
| 24 | pC-5′UTR- | AGGGGGTGGGC CTATAGACTCTA TAGGCGGTACTT |
| | Reverse 3 | ACGTCACTCTTG GCACGGGGAAT CC |
| | primer | |
| 25 | pC-5′UTR- | CACCAACTAGTG CATAAGAAGCC AAGGGGGTGGG |
| | Reverse 4 | CCTATAGACTC |
| | primer | |

PCR reactions were performed using AccuPrime pfx DNA polymerase (Invitrogen, 12344), following the manufacturer's protocol. The primers were used in a series of reactions detailed in Table 6 below to progressively add sequence from the native 5′ UTR sequence of CMV downstream of the CMV promoter sequence. Each reaction product was gel purified and then used as the template for the next reaction.

TABLE 7

| | Forward | Reverse | |
|---|---|---|---|
| Reaction | Primer | Primer | Template |
| 1 | pC forward | pCMV3′ end of | pC |
| | primer with | 5′UTR reverse | |
| | Pac1 site | primer | |
| 2 | pC forward | pC-5′UTR- | Product |
| | primer with | Reverse 1 | from |
| | Pac1 site | | Reaction 1 |
| 3 | pC forward | pC-5′UTR- | Product |
| | primer with | Reverse 2 | from |
| | Pac1 site | | Reaction 2 |
| 4 | pC forward | pC-5′UTR- | Product |
| | primer with | Reverse 3 | from |
| | Pac1 site | | Reaction 3 |
| 5 | pC forward | pC-5′UTR- | Product |
| | primer with | Reverse 4 | from |
| | Pac1 site | | Reaction 4 |

Construction of CMV Promoter with gpd 5′ UTR.

Pairs of primers, shown in the Table below, were used to amplify CMV promoter sequences with overlapping sequence to each of the gpd 5′ UTR fragments (100 bp gpd 5′ UTR, 150 bp gpd 5′ UTR, and 200 bp gpd 5′ UTR). The template DNA was the pC-BG vector described above in Example 1.

TABLE 8

| SEQ | | |
|---|---|---|
| ID | | |
| NO: | Description | Sequence |
| 20 | pC forward | CACCATTAATTAAGTCGTTACATAACTTACGGTAAATGG |
| | primer with | |
| | PacI site | |
| 26 | pC overlap- | GCGAACAGAAGCCGCATCGTACGGTTCACTAAACGAGCTC |
| | 100 bp gpd 5′ | |
| | UTR reverse | |
| | primer | |
| 27 | pC overlap- | GAGAGTCTGGCGGGGTAGCTACGGTTCACTAAACGAGCTC |
| | 150 bp gpd 5′ | |
| | UTR reverse | |
| | primer | |
| 28 | pC overlap- | GCGAACAGAAGCCGCATCGTACGGTTCACTAAACGAGCTC |
| | 200 bp gpd 5′ | |
| | UTR reverse | |
| | primer | |

The gpd 5′ UTR fragments were amplified from pWG, containing a fragment of the gpd gene upstream of the translational start cloned into pW (described in Example 1 above), using a forward primer specific to each gpd 5′ UTR fragment (100 bp, 150 bp or 200 bp, respectively) and a single reverse primer. Forward and reverse primers were as follows.

TABLE 9

| SEQ | | |
|---|---|---|
| ID | | |
| NO: | Description | Sequence |
| 29 | pC overlap- | GAGCTCGTTTAGTGAACCGTACGATGCGGCTTCTGTTCGC |
| | 100 bp gpd 5′ | |
| | UTR forward | |
| | primer | |
| 30 | pC overlap- | GAGCTCGTTTAGTGAACCGTAGCTACCCCGCCAGACTCTC |
| | 150 bp gpd 5′ | |
| | UTR forward | |
| | primer | |
| 31 | pC overlap- | GAGCTCGTTTAGTGAACCGTACGATGCGGCTTCTGTTCGC |
| | 200 bp gpd 5′ | |
| | UTR forward | |
| | primer | |
| 32 | pWG-SpeI | CACCAACTAGTTTTGTATCTGCGAATTGAGCTTGCGTGA |
| | site reverse | |
| | primer | |

By pairing each forward primer with the reverse primer, 5′ UTR fragments were generated that included 100 bp, 150 bp, or 200 bp fragments from the 5′ UTR of gpd as well as sequence overlapping with the amplified CMV promoter fragments described above, such that resulting CMV promoter and 5′UTR fragments could readily be ligated together for subcloning.

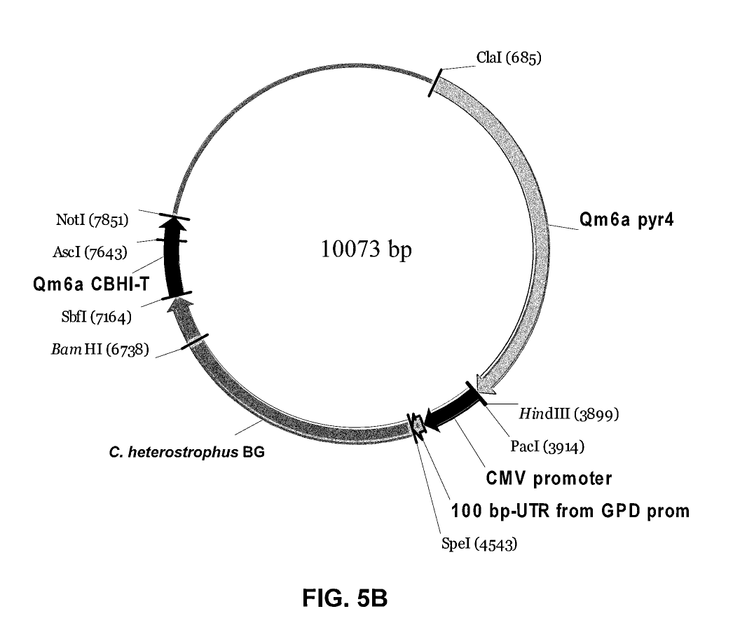

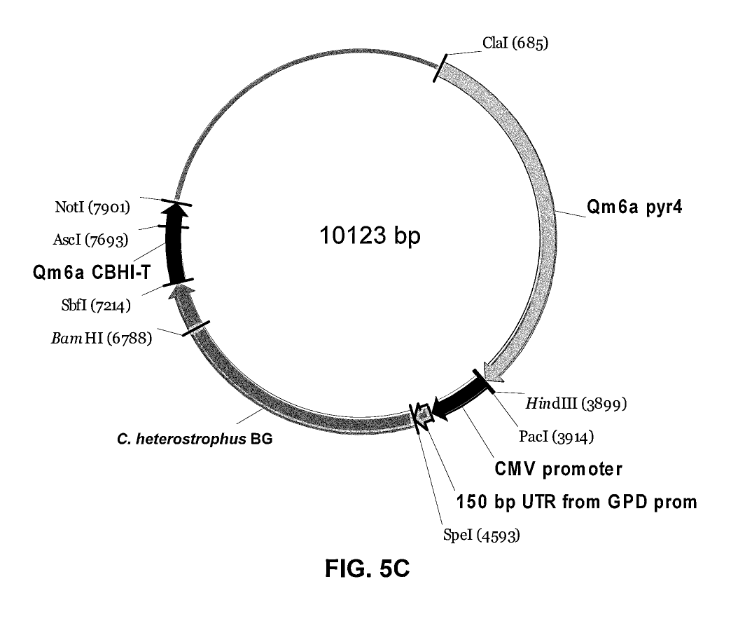

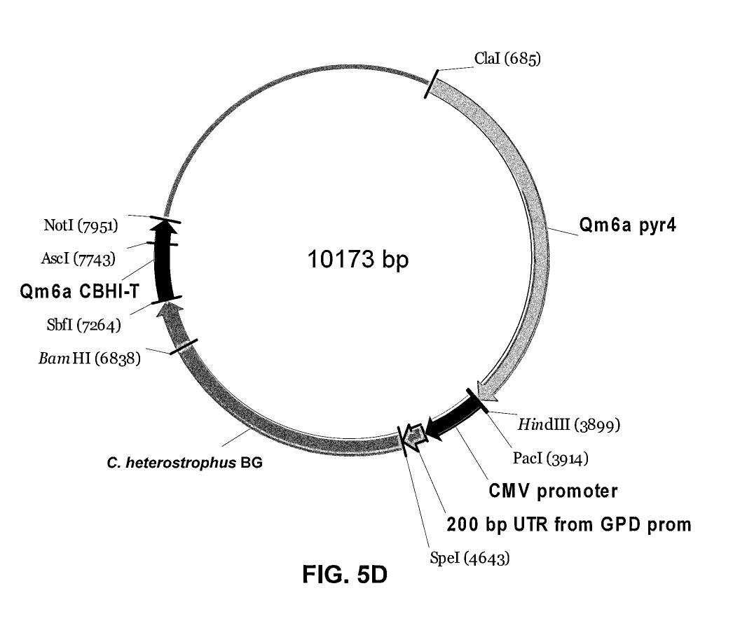

PCR reactions were performed by using AccuPrime pfx DNA polymerase (Invitrogen, 12344) and following manufacturer's protocol. The resulting DNA fragments containing promoter and 5′ UTR sequences were subcloned as follows into the pC vector. The PCR products were purified by Zymoclean Gel DNA Recovery kit (Zymo Research, D4001). Purified PCR fragments and pC DNA were digested with restriction enzymes Pac I (New England Biolabs R0547S) and Spe I (New England Biolabs R0133S) to create cloning ends. pC vector and PCR insert were ligated by T4 DNA ligase (Roche, 11 635 379 001) and transformed E. coli competent cells XL1-Blue (Stratagene, 200236) following manufactures' instructions, generating vectors containing expression cassettes comprising a CMV promoter, a 5′ UTR sequence, a protein coding sequence, and a terminator sequence. The vectors, schematically represented in FIG. 5, are denominated as follows: pC-5′UTR for an expression cassette containing a 5′UTR from the CMV native 5′UTR (FIG. 5A), and pC-100 (FIG. 5B), pC-150 (FIG. 5C), and pC-200 (FIG. 50) for expression cassettes containing a 100 nucleotide sequence (SEQ ID NO:2), 150 nucleotide sequence (SEQ ID NO:3), and 200 nucleotide sequence (SEQ ID NO:4), of the 5′UTR of the gpd gene, respectively.

Transformation.

Each of the expression cassettes was transformed into Trichoderma reesei according to the protocol described above in Example 3. Specifically, protoplasts of the strain Trichoderma reesei MCG80 pyr4- were prepared as described above, and used in transformations with each one of the eight constructs described in the previous section containing a UTR sequence downstream of the viral promoter in each case, but upstream of the β-glucosidase coding sequence.

### 5.5. Example 5

### β-Glucosidase Activity in Trichoderma Reesei Transformants Containing CMV-5′UTR or CMV Expression Cassettes

This example provides a demonstration of β-glucosidase activity in T reesei transformants containing CMV-5′UTR or CMV expression cassettes, showing the increase in enzyme activity in Trichoderma reesei strains transformed with a vector comprising a full expression cassette as compared to vectors containing a promoter operably linked to a protein coding sequence.

Growth Conditions and Media.

For analysis of expression among Trichoderma reesei transformants, individual isolates displaying the pyre phenotype were inoculated into the wells of a 96-well plate containing 0.2 ml/well ACM (Aspergillus Complete Medium) or CM (Complete Medium). Complete medium was as follows: 0.5% yeast extract, 1% glucose (filtered), 0.2% casamino acids (sterile), 7 mM KCl; 11 mM KH₂PO₄; 70 mM NaNO₃; 2 mM MgSO₄; 77 μM ZnSO₄; 178 μM H₃BO₃; 25 μM MnCl₂; 18 μM FeSO₄; 7.1 μM CoCl₂; 6.4 μM CuSO₄; 6.2 μM Na₂MoO₄; 134 μM Na₂EDTA; 1 mg/ml riboflavin; 1 mg/ml thiamine; 1 mg/ml nicotinamide; 0.5 mg/ml pyridoxine; 0.1 mg/ml pantothenic acid; 2 μg/ml biotin; 1 mM uridine (filtered). Plates were incubated in a stationary, humidified incubator at 30° C. for 7 days. Following incubation, the fluid underneath the fungal mats was harvested and assayed for β-glucosidase activity as follows.

β-Glucosidase Activity Assay.

The β-glucosidase activities of harvested fluid samples were measured using 4MU-G (Sigma product#M3633) as substrate in an assay performed on liquid handling robot. The method is as follows: 100 μl aliquots of reaction buffer (0.5 mM 4MU-G in 100 mM NaOAc, pH5.0) were transferred into each well of a 96-well flat-bottom microplate (Corning Inc., Costar, black polystyrene) using a Titertek Multidrop mircroplate dispenser (Titertek, Huntsville, Ala.). The reactions were then initiated by the addition of 4 μl aliquots of the harvested fluid samples, transferred and mixed on a VPrep pipetting system (Agilent, Santa Clara, Calif.). The microplate containing the reaction buffer and samples was then incubated at room temperature for 3 minutes. After incubation, the reaction was stopped by the addition of 100 μl aliquots of stop buffer (400 mM Sodium Carbonate, pH10.0) into each well using a Titertek Multidrop microplate dispenser. The fluorescence of each well was then measured as relative units at 360/465 nm (denoted RFU) using an Ultra Microplate Reader (Tecan Group Ltd., Männedorf Switzerland). The relative fluorescence of the transformants were then compared to the RFU signals of the control, untransformed strains.

Results.

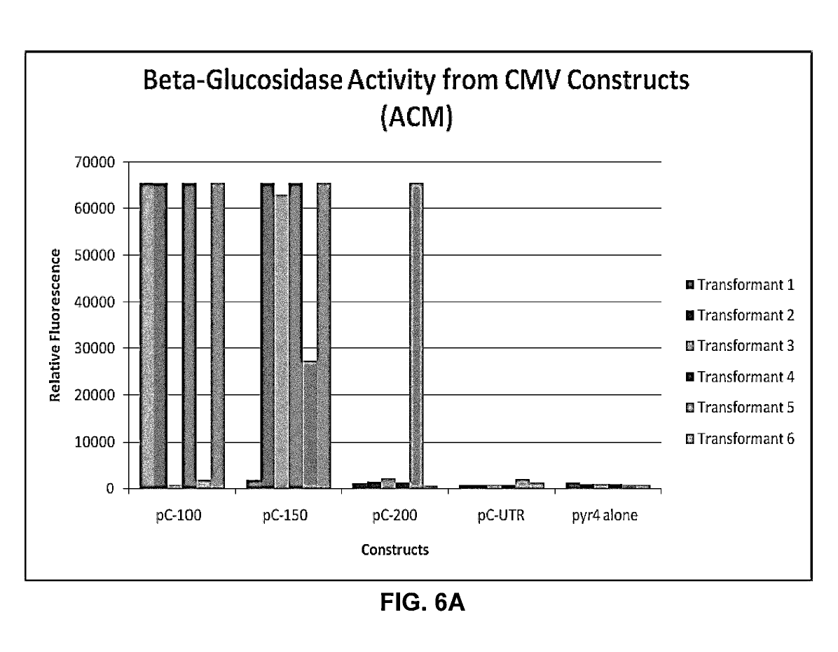

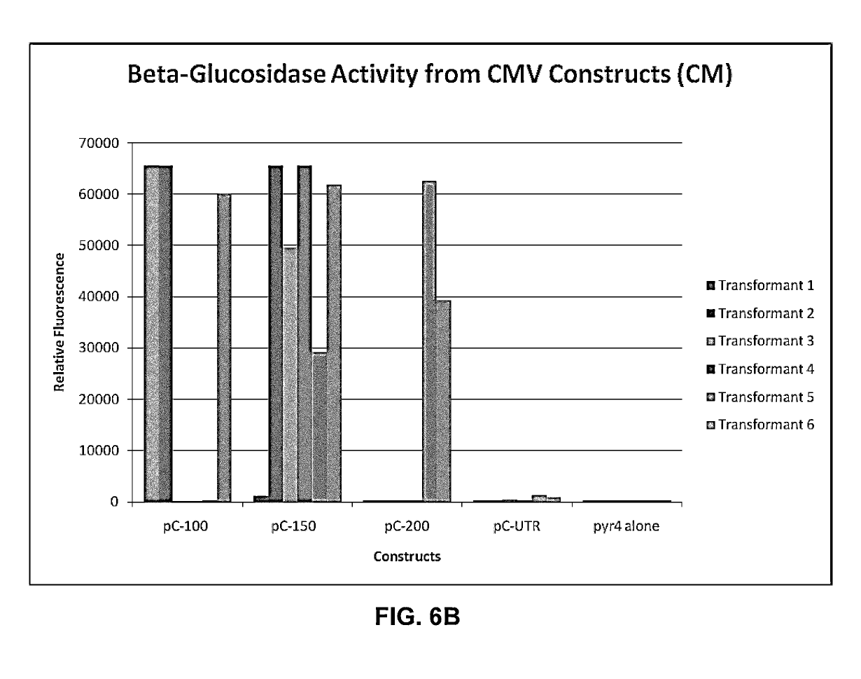

β-glucosidase activity from transformants containing a vector bearing the pC-BG was not significantly above background. FIG. 6A-B provides bar charts of β-glucosidase activity in Trichoderma reesei transformants bearing a 5′ untranslated region from the native Trichoderma reesei gpd gene, or the native CMV viral gene in addition to the CMV promoter relative to control, untransformed Trichoderma reesei tested in ACM (FIG. 6A) or CM (FIG. 6B). The constructs containing expression cassettes bearing a CMV promoter and a 5′ untranslated region from the native Trichoderma reesei gpd gene showed expression significantly above the background level of activity generated by the native T reesei β-glucosidase activity. Thus, expression cassettes comprising a mammalian viral promoter, a 5′ UTR operable in the filamentous fungal strain, a protein coding sequence, and a terminator sequence comprising a 3′ UTR result in efficient translation of the transcript leading to increased activity of a protein.

### 5.6. Example 6

### Fermentation of T. reesei Strains Containing a CMV Promoter Construct

This example provides a demonstration that the expression cassettes of the disclosure can be used for fermentative production of recombinant polypeptides.

A T reesei production strain containing a single stably-integrated copy of the construct described in Example 4 (pC-200) was grown in fed-batch fermentations in 40 L fermenters using the following procedure, alongside a non-recombinant production strain as a control.

Seed flasks were inoculated with samples of mycelial stocks of each of the two strains (0.5 ml stock into 200 mL media in baffled 2 L flasks). The seed media was composed of: standard salts medium enriched with complex nitrogen, glucose and Trace Element solution; water added to a volume of 200 mL, media was autoclaved for 30 minutes at 122° C. The shake flasks were incubated in a shaking incubator at 31° C. and 220 rpm. The OD600 was measured at 24 hours and at 6-hour intervals thereafter. When the OD600 reached approximately 5.0, 60 ml of the culture was transferred to a seed tank.

The seed tank contained 15 L media in a 30 L fermenter. The seed tank media was composed of standard salts medium enriched with glucose, hemicellulose, cellulose, and Trace Element solution; water added to a final volume of 15 L. The media was sterilized in place at 122° C. for 60 minutes and cooled prior to inoculation. The fermentation culture was grown at 25° C., pH 4.2, 20 LPM air flow, and a dissolved oxygen (DO) set point of 20%, with agitation cascading from 100-800 rpm to maintain DO. Samples of the fermentation culture were taken every 6 hours and measured for OD600 and residual glucose concentration. Once the OD600 of each strain reached 45-55, 1.3 L was transferred to a 40 L fermenter representing the main fermenter for the experiment.

The main fermentation tank contained initially 10 L of base medium. The base medium was composed of standard salts medium enriched with glucose, hemicellulose, cellulose, and Trace Element solution; water added to a final volume of 10 L, then sterilized in place at 122° C. for 60 minutes. The fermentation set points used were as follows: 25° C., 20% DO, agitation cascading from 100-800 rpm to maintain DO, pH 4.5, air flow starting at 10 LPM and rising to 15 LPM when agitation reached 800 rpm. Nutrient feed was added according to a pre-determined feed profile starting at 1.3 mL/min and rising to 4 mL/min. The nutrient feed media was composed of standard salts medium enriched with glucose, hemicellulose, cellulose, lactose and Trace Element solution; water added to a final volume of 1 L, then sterilized at 122° C. for 60 minutes. After 48 hours of fermentation, samples from each fermenter were at 24-hour intervals. The samples were centrifuged to separate cell mass from supernatant and the supernatant assayed for β-glucosidase activity using 4-nitrophenyl β-D-glucuronide (pNP-G) as substrate.

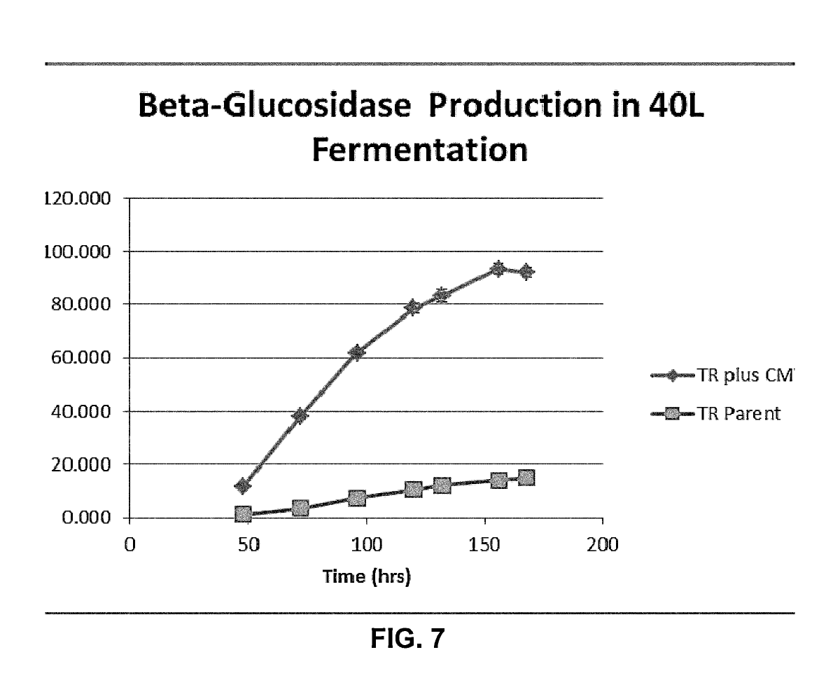

Results: As shown in FIG. 7, the production of β-glucosidase activity in the supernatant is approximately five times higher in the recombinant strain containing the Cochliobolus heterostrophus β-glucosidase transcribed from the CMV promoter than the activity shown by the native β-glucosidase produced by the parent production strain under similar conditions. The Cochliobolus β-glucosidase and the native β-glucosidase show approximately the same specific activity on the pNP-G substrate.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

What is claimed is:

1. A nucleic acid comprising an expression cassette, said expression cassette comprising, operably linked in a 5′ to 3′ direction:
(a) a mammalian viral promoter;
(b) a 5′ untranslated region (“UTR”) operable in filamentous fungi;
(c) a first protein coding sequence comprising a start codon and a stop codon; and
(d) a 3′ UTR.

2. The nucleic acid of claim 1, wherein the 5′ UTR is operable in T reesei.

3. The nucleic acid of claim 1, wherein the mammalian viral promoter is a Rous sarcoma virus (RSV) long terminal repeat (LTR) promoter, a cytomegalovirus immediate early gene (CMV) promoter, a simian virus early (SV40) promoter, or an adenovirus major late promoter.

4. The nucleic acid of claim 1, wherein the promoter is not an SV40 promoter.

5. The nucleic acid of claim 3, wherein the mammalian viral promoter is a CMV promoter.

6. The nucleic acid of claim 5, wherein the CMV promoter comprises the nucleotide sequence of SEQ ID NO:1.

7. The nucleic acid of claim 1, wherein the 5′ UTR is from the Trichoderma reesei glyceraldehyde-3-phosphate dehydrogenase gene.

8. The nucleic acid of claim 7, wherein the 5′ UTR comprises the nucleotide sequence of SEQ ID NO:2.

9. The nucleic acid of claim 8, wherein the 5′ UTR comprises the nucleotide sequence of SEQ ID NO:3.

10. The nucleic acid of claim 1, wherein the 3′ UTR comprises a polyadenylation signal.

11. The nucleic acid of claim 1, which further comprises between the first protein coding sequence and the 3′ UTR an internal ribosome entry site (“IRES”) and a second protein coding sequence.

12. The nucleic acid of claim 1, wherein the first protein is a filamentous fungal protein.

13. The nucleic acid of claim 12, wherein the first protein is a Trichoderma reesei protein.

14. The nucleic acid of claim 1, wherein the first protein is a yeast, mammalian or bacterial protein.

15. The nucleic acid of claim 1, wherein the first protein is a β-glucosidase.

16. The nucleic acid of claim 15, wherein the β-glucosidase comprises the amino acid sequence of SEQ ID NO:34.

17. The nucleic acid of claim 1, wherein the first protein comprises a signal sequence.

18. The vector comprising the nucleic acid of claim 1.

19. The vector of claim 18 which comprises an origin of replication.

20. The vector of claim 18 which comprises a selectable marker.

21. The vector of claim 20, wherein the selectable marker is an antibiotic resistance gene or an auxotrophic marker.

22. A filamentous fungal cell comprising a recombinant expression cassette, said expression cassette comprising:
(a) a mammalian viral promoter;
(b) a 5′ untranslated region (“UTR”) operable in said filamentous fungus;
(c) a first protein coding sequence comprising a start codon and a stop codon; and
(d) a 3′ UTR.

23. The filamentous fungal cell of claim 22, wherein the mammalian viral promoter is a RSV LTR promoter, a CMV promoter, an SV40 promoter, or an adenovirus major late promoter.

24. The filamentous fungal cell of claim 22, wherein the mammalian viral promoter is not the SV40 promoter.

25. The filamentous fungal cell of claim 23, wherein the mammalian viral promoter is a CMV promoter.

26. The filamentous fungal cell of claim 25, wherein the CMV promoter comprises the nucleotide sequence of SEQ ID NO:1.

27. The filamentous fungal cell of claim 22, wherein the 5′ UTR and/or the 3′ UTR is native to the filamentous fungal cell.

28. The filamentous fungal cell of claim 22, wherein the 5′ UTR is from the Trichoderma reesei glyceraldehyde-3-phosphate dehydrogenase gene.

29. The filamentous fungal cell of claim 28, wherein the 5′ UTR comprises the nucleotide sequence of SEQ ID NO:2.

30. The filamentous fungal cell of claim 29, wherein the 5′ UTR comprises the nucleotide sequence of SEQ ID NO:3.

31. The filamentous fungal cell of claim 22, wherein the 3′ UTR comprises a polyadenylation signal.

32. The filamentous fungal cell of claim 22, wherein the first protein coding sequence is native to the filamentous fungal cell.

33. The filamentous fungal cell of claim 22, wherein the expression cassette further comprises between the first protein coding sequence and the 3′ UTR an internal ribosome entry site (“IRES”) and a second protein coding sequence.

34. The filamentous fungal cell of claim 33, wherein the second protein coding sequence is 5′ to the first protein coding sequence.

35. The filamentous fungal cell of claim 33, wherein the second protein coding sequence is 3′ to the first protein coding sequence.

36. The filamentous fungal cell of claim 22, wherein the first protein is a filamentous fungal protein.

37. The filamentous fungal cell of claim 36, wherein the first protein is a Trichoderma reesei protein.

38. The filamentous fungal cell of claim 22, wherein the first protein is a yeast, mammalian or bacterial protein.

39. The filamentous fungal cell of claim 22, wherein the first protein coding sequence encodes a β-glucosidase.

40. The filamentous fungal cell of claim 39, wherein the β-glucosidase comprises the amino acid sequence of SEQ ID NO:34.

41. The filamentous fungal cell of claim 22, wherein the expression cassette is in the filamentous fungal cell genome.

42. The filamentous fungal cell of claim 22, wherein the expression cassette is on an extragenomic vector.

43. The filamentous fungal cell of claim 42, wherein the extragenomic plasmid is a vector comprises a selectable marker.

44. The filamentous fungal cell of claim 22, which is a which is a species of Acremonium, Aspergillus, Emericella, Fusarium, Humicola, Mucor, Myceliophthora, Neurospora, Penicillium, Scytalidium, Thielavia, Chrysosporium, Phanerochaete, Tolypocladium, or Trichoderma.

45. The filamentous fungal cell of claim 44, which is of the species Aspergillus awamori, Aspergillus fumigatus, Aspergillus foetidus, Aspergillus japonicus, Aspergillus nidulans, Aspergillus niger, Aspergillus oryzae, Chrysosporium lucknowense, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Neurospora intermedia, Penicillium purpurogenum, Penicillium canescens, Penicillium solitum, Penicillium funiculosum, Phanerochaete chrysosporium, Phlebia radiate, Pleurotus eryngii, Thielavia terrestris, Trichoderma harzianum, Trichoderma longibrachiatum, Trichoderma reesei, or Trichoderma viride.

46. The filamentous fungal cell of claim 44, which is not an Aspergillus flavus.

47. The filamentous fungal cell of claim 44, which is not an Aspergillus.

48. The filamentous fungal cell of claim 44, which is a Trichoderma reesei.

49. The filamentous fungal cell of claim 22, wherein the first protein coding sequence encodes a protein comprising a signal sequence.

50. The filamentous fungal cell of claim 22, wherein the first protein is a cellulase, a hemicellulase or an accessory protein.

51. The filamentous fungal cell of claim 50, wherein the cellulase, hemicellulase or accessory protein comprises a signal sequence.

52. The filamentous fungal cell of claim 33, wherein the second protein is a cellulase, a hemicellulase or an accessory protein.

53. The filamentous fungal cell of claim 52, wherein the cellulase, hemicellulase or accessory protein comprises a signal sequence.

54. A method for producing a recombinant protein, comprising culturing the filamentous fungal cell of claim 22 under conditions that result in expression of the first protein.

55. The method of claim 54, further comprising recovering the first protein.

56. The method of claim 55, further comprising purifying the first protein.

57. A method for producing a secreted protein, comprising culturing the filamentous fungal cell of claim 49 under conditions that result in expression and secretion of the first protein.

58. The method of claim 57, further recovering the first protein.

59. The method of claim 58, wherein the first protein is recovered from the culture medium.

60. The method of claim 59, further comprising purifying the first protein.

61. A method for producing a cellulase composition, comprising culturing the filamentous fungal cell of claim 50 under conditions that result in expression of the first protein.

62. The method of claim 61, further comprising recovering a cellulase composition.

63. The method of claim 62, wherein the cellulase composition is a fermentation broth in which the filamentous fungal cells are cultured.

64. A method for producing a cellulase composition, comprising culturing the filamentous fungal cell of claim 52 under conditions that result in expression of the second protein.

65. The method of claim 64, further comprising recovering a cellulase composition.

66. The method of claim 65, wherein the cellulase composition is a fermentation broth in which the filamentous fungal cells are cultured.

67. A method for saccharifying biomass, comprising:
(a) producing a cellulase composition by the method of claim 61;
(b) treating biomass with said cellulase composition, thereby producing saccharifying said biomass.

68. The method of claim 67, further comprising recovering fermentable sugars from said saccharified biomass.

69. The method of claim 68, wherein the fermentable sugars comprise disaccharides.

70. The method of claim 68, wherein the fermentable sugars comprise monosaccharides.

71. The method of claim 67, wherein said biomass is corn stover, bagasses, sorghum, giant reed, elephant grass, miscanthus, Japanese cedar, wheat straw, switchgrass, hardwood pulp, softwood pulp, crushed sugar cane, energy cane, or Napier grass.

72. The method of claim 67, further comprising, prior to step (b), pretreating the biomass.

73. A method for producing a fermentation product, comprising:
(a) producing a cellulase composition by the method of claim 61;
(b) treating biomass with said cellulase composition, thereby producing fermentable sugars; and
(c) culturing a fermenting microorganism in the presence of the fermentable sugars produced in step (b) under fermentation conditions, thereby producing a fermentation product.

74. The method of claim 73, wherein said fermentable sugars comprise disaccharides.

75. The method of claim 73, wherein the fermentable sugars comprise monosaccharides.

76. The method of claim 73, wherein the fermentation product is ethanol.

77. The method of claim 73, further comprising, prior to step (b), pretreating the biomass.

78. The method of claim 73, wherein said fermenting microorganism is a bacterium or a yeast.

79. The method of claim 73, wherein said fermenting microorganism is a bacterium selected from Zymomonas mobilis, Escherichia coli and Klebsiella oxytoca.

80. The method of claim 73, wherein said fermenting microorganism is a yeast selected from Saccharomyces cerevisiae, Saccharomyces uvarum, Kluyveromyces fragilis, Kluyveromyces lactis, Candida pseudotropicalis, and Pachysolen tannophilus.

81. The method of claim 73, wherein said biomass is corn stover, bagasses, sorghum, giant reed, elephant grass, miscanthus, Japanese cedar, wheat straw, switchgrass, hardwood pulp, softwood pulp, crushed sugar cane, energy cane, or Napier grass.


SEQUENCE LISTING (34 sequences)

SEQ ID NO: 1 - DNA, 525 bp, Cytomegalovirus
gtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgt

SEQ ID NO: 2 - DNA, 100 bp, Trichoderma reesei
cctcctccctctctccctctcgtttcttcctaacaaacaaccaccaccaaaatctctttggaagctcacgactcacgcaagctcaattcgcagatacaaa

SEQ ID NO: 3 - DNA, 150 bp, Trichoderma reesei
agctaccccgccagactctcctgcgtcaccaatttttttccctatttacccctcctccctctctccctctcgtttcttcctaacaaacaaccaccaccaaaatctctttggaagctcacgactcacgcaagctcaattcgcagatacaaa

SEQ ID NO: 4 - DNA, 200 bp, Trichoderma reesei
acgatgcggcttctgttcgcctgcccctcctcccactcgtgcccttgacgagctaccccgccagactctcctgcgtcaccaatttttttccctatttacccctcctccctctctccctctcgtttcttcctaacaaacaaccaccaccaaaatctctttggaagctcacgactcacgcaagctcaattcgcagatacaaa

SEQ ID NO: 5 - DNA, 688 bp, Trichoderma reesei
ggccaggtcctgaacccttactactctcagtgcctgtaaagctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaacaaacatcccgttcataacccgtagaatcgccgctcttcgtgtatcccagtaccacggcaaaggtatttcatgatcgttcaatgttgatattgttcccgccagtatggctccacccccatctccgcgaatctcctcttctcgaacgcggtagtggcgcgccaattggtaatgacccatagggagacaaacagcataatagcaacagtggaaattagtggcgcaataattgagaacacagtgagaccatagctggcggcctggaaagcactgttggagaccaacttgtccgttgcgaggccaacttgcattgctgtcaggacgatgacaacgtagccgaggaccgtcacaagggacgcaagtgcg

SEQ ID NO: 6 - DNA, 42 bp, Cytomegalovirus
caccattaattaagtcgttacataacttacggtaaatggccc

SEQ ID NO: 7 - DNA, 42 bp, Cytomegalovirus
caccacggaccgtactagtacggttcactaaacgagctctgc

SEQ ID NO: 8 - DNA, 39 bp, Cochliobolus heterostrophus
caccaactagtatgctgtggcttgcacaagcattgttgg

SEQ ID NO: 9 - DNA, 42 bp, Cochliobolus heterostrophus
caccaggccggccttatctaaagctgctagtgtccagtgggg

SEQ ID NO: 10 - DNA, 21 bp, Trichoderma reesei
actttgcgtcccttgtgacgg

SEQ ID NO: 11 - DNA, 21 bp, Trichoderma reesei
ttgcattggtacagctgcagg

SEQ ID NO: 12 - DNA, 21 bp, Trichoderma reesei
gactcacgcaagctcaattcg

SEQ ID NO: 13 - DNA, 23 bp, Trichoderma reesei
ccagactctcctgcgtcaccaat

SEQ ID NO: 14 - DNA, 24 bp, Trichoderma reesei
ctacaatcatcaccacgatgctcc

SEQ ID NO: 15 - DNA, 24 bp, Trichoderma reesei
cgacattctctcctaatcaccagc

SEQ ID NO: 16 - DNA, 24 bp, Trichoderma reesei
gccgtgcctacctgctttagtatt

SEQ ID NO: 17 - DNA, 24 bp, Trichoderma reesei
ccactatctcaggtaaccaggtac

SEQ ID NO: 18 - DNA, 24 bp, Trichoderma reesei
gtctcgctccacttgatgttggca

SEQ ID NO: 19 - DNA, 170 bp, Cytomegalovirus
cagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgc

SEQ ID NO: 20 - DNA, 39 bp, Cytomegalovirus
caccattaattaagtcgttacataacttacggtaaatgg

SEQ ID NO: 21 - DNA, 39 bp, Cytomegalovirus; identical to SEQ ID NO: 20

SEQ ID NO: 22 - DNA, 60 bp, Cytomegalovirus
cggccgcggaggctggatcggtcccggtgtcttctatggaggtcaaaacagcgtggatgg

SEQ ID NO: 23 - DNA, 60 bp, Cytomegalovirus
actcttggcacggggaatccgcgttccaatgcaccgttcccggccgcggaggctggatcg

SEQ ID NO: 24 - DNA, 60 bp, Cytomegalovirus
agggggtgggcctatagactctataggcggtacttacgtcactcttggcacggggaatcc

SEQ ID NO: 25 - DNA, 45 bp, Cytomegalovirus
caccaactagtgcataagaagccaagggggtgggcctatagactc

SEQ ID NO: 26 - DNA, 40 bp, Unknown
  CMV/T.reesei
gcgaacagaagccgcatcgtacggttcactaaacgagctc

SEQ ID NO: 27 - DNA, 40 bp, Unknown
  CMV/T.reesei
gagagtctggcggggtagctacggttcactaaacgagctc

SEQ ID NO: 28 - DNA, 40 bp, Unknown; identical to SEQ ID NO: 26
  CMV/T.reesei

SEQ ID NO: 29 - DNA, 40 bp, Unknown
  CMV/T.reesei
gagctcgtttagtgaaccgtacgatgcggcttctgttcgc

SEQ ID NO: 30 - DNA, 40 bp, Unknown
  CMV/T.reesei
gagctcgtttagtgaaccgtagctaccccgccagactctc

SEQ ID NO: 31 - DNA, 40 bp, Unknown; identical to SEQ ID NO: 29
  CMV/T.reesei

SEQ ID NO: 32 - DNA, 39 bp, Trichoderma reesei
caccaactagttttgtatctgcgaattgagcttgcgtga

SEQ ID NO: 33 - DNA, 2610 bp, Cochliobolus heterostrophus
atgctgtggcttgcacaagcattgttggtcggccttgcccaggcatcgcccaggttccctcgtgctaccaacgacaccggcagtgattctttgaacaatgcccagagcccgccattctacccaagtccttgggtagatcccaccaccaaggactgggcggctgcctatgaaaaagcaaaggcttttgttagccaattgactcttattgagaaggtcaacctcaccaccggcactggatggcagagcgaccactgcgttggtaacgtgggcgctattcctcgccttggctttgatcccctctgcctccaggacagccctctcggcatccgtttcgcagactacgtttctgctttcccagcaggtggcaccattgctgcatcatgggaccgctatgagttttacacccgcggtaacgagatgggtaaggagcaccgaaggaagggagtcgacgttcagcttggtcctgccattggacctcttggtcgccaccccaagggcggtcgtaactgggaaggcttcagtcctgatcctgtactttccggtgtggccgtgagcgaaacagtccgcggtatccaggatgctggtgtcattgcctgcactaagcacttccttctgaacgagcaagaacatttccgtcagcccggcagtttcggagatatcccctttgtcgatgccatcagctccaataccgatgacacgactctacacgagctctacctgtggccctttgccgacgccgtccgcgctggtactggtgccatcatgtgctcttacaacaaggccaacaactcgcaactctgccaaaactcgcaccttcaaaactatattctcaagggcgagcttggcttccagggtttcattgtatctgactgggatgcacagcactcgggcgttgcgtcggcttatgctggattggacatgactatgcctggtgatactggattcaacactggactgtccttctggggcgctaacatgaccgtctccattctcaacggcaccattccccagtggcgtctcgacgatgcggccatccgtatcatgaccgcatactactttgtcggccttgatgagtctatccctgtcaactttgacagctggcaaactagcacgtacggattcgagcattttttcggaaagaagggcttcggtctgatcaacaagcacattgacgttcgcgaggagcacttccgctccatccgccgctctgctgccaagtcaaccgttctcctcaagaactctggcgtccttcccctctctggaaaggagaagtggactgctgtatttggagaagatgctggcgaaaacccgctgggccccaacggatgcgctgaccgcggctgcgactctggcaccttggccatgggctggggttcgggaactgcagacttcccttacctcgtcactcctctcgaagccatcaagcgtgaggttggcgagaatggcggcgtgatcacttcggtcacagacaactacgccacttcgcagatccagaccatggccagcagggccagccactcgattgtcttcgtcaatgccgactctggtgaaggttacatcactgttgataacaacatgggtgaccgcaacaacatgactgtgtggggcaatggtgatgtgcttgtcaagaatatctctgctctgtgcaacaacacgattgtggttatccactctgtcggcccagtcattattgacgcctggaaggccaacgacaacgtgactgccattctctgggctggtcttcctggccaggagtctggtaactcgattgctgacattctatacggacaccacaaccctggtggcaagctccccttcaccattggcagctcttcagaggagtatggccctgatgtcatctacgagcccacgaacggcatcctcagccctcaggccaactttgaagagggcgtcttcattgactaccgcgcgtttgacaaggcgggcattgagcccacgtacgaatttggctttggtctttcgtacacgacttttgaatactcggacctcaaggtcactgcgcagtctgccgaggcttacaagcctttcaccggccagacttcggctgcccctacattcggaaacttcagcaagaaccccgaggactaccagtaccctcccggccttgtttaccccgacacgttcatctacccctacctcaactcgactgacctcaagacggcatctcaggatcccgagtacggcctcaacgttacctggcccaagggctctaccgatggctcgcctcagacccgcattgcggctggtggtgcgcccggcggtaacccccagctctgggacgttttgttcaaggtcgaggccacgatcaccaacactggtcacgttgctggtgacgaggtggcccaggcgtacatctcgcttggtggccccaacgaccccaaggtgctactccgtgactttgaccgcttgaccatcaagcctggtgagagcgctgttttcacagccaacatcacccgccgtgatgtcagcaactgggacactgtcagccagaactgggtcattaccgagtaccccaagacgatccacgttggtgccagttcgaggaaccttcctctttctgccccactggacactagcagctttagataa

SEQ ID NO: 34 - protein, 869 aa, Cochliobolus heterostrophus
MLWLAQALLVGLAQASPRFPRATNDTGSDSLNNAQSPPFYPSPWVDPTTKDWAAAYEKAKAFVSQLTLIEKVNLTTGTGWQSDHCVGNVGAIPRLGFDPLCLQDSPLGIRFADYVSAFPAGGTIAASWDRYEFYTRGNEMGKEHRRKGVDVQLGPAIGPLGRHPKGGRNWEGFSPDPVLSGVAVSETVRGIQDAGVIACTKHFLLNEQEHFRQPGSFGDIPFVDAISSNTDDTTLHELYLWPFADAVRAGTGAIMCSYNKANNSQLCQNSHLQNYILKGELGFQGFIVSDWDAQHSGVASAYAGLDMTMPGDTGFNTGLSFWGANMTVSILNGTIPQWRLDDAAIRIMTAYYFVGLDESIPVNFDSWQTSTYGFEHFFGKKGFGLINKHIDVREEHFRSIRRSAAKSTVLLKNSGVLPLSGKEKWTAVFGEDAGENPLGPNGCADRGCDSGTLAMGWGSGTADFPYLVTPLEAIKREVGENGGVITSVTDNYATSQIQTMASRASHSIVFVNADSGEGYITVDNNMGDRNNMTVWGNGDVLVKNISALCNNTIVVIHSVGPVIIDAWKANDNVTAILWAGLPGQESGNSIADILYGHHNPGGKLPFTIGSSSEEYGPDVIYEPTNGILSPQANFEEGVFIDYRAFDKAGIEPTYEFGFGLSYTTFEYSDLKVTAQSAEAYKPFTGQTSAAPTFGNFSKNPEDYQYPPGLVYPDTFIYPYLNSTDLKTASQDPEYGLNVTWPKGSTDGSPQTRIAAGGAPGGNPQLWDVLFKVEATITNTGHVAGDEVAQAYISLGGPNDPKVLLRDFDRLTIKPGESAVFTANITRRDVSNWDTVSQNWVITEYPKTIHVGASSRNLPLSAPLDTSSFR